(19) United States
(12) Patent Application Publication
(10) Pub. No.: US 2021/0287777 A1
(43) Pub. Date: Sep. 16, 2021

(54) HEALTH TRACKING SYSTEMS AND METHODS FOR GEOLOCATION-BASED RESTAURANT MATCHING

(71) Applicant: Cambia Health Solutions, Inc., Portland, OR (US)

(72) Inventors: Alexander Thorpe, Beaverton, OR (US); Max Janasik, Portland, OR (US); Stephen Erdmann, Lake Oswego, OR (US); Mohan Nair, Lake Oswego, OR (US); Steve Warming, Beaverton, OR (US); Marcie Traylor, Portland, OR (US)

(21) Appl. No.: 17/336,227

(22) Filed: Jun. 1, 2021

Related U.S. Application Data
(63) Continuation-in-part of application No. 14/856,430, filed on Sep. 16, 2015.
(60) Provisional application No. 62/051,786, filed on Sep. 17, 2014.

(51) Int. Cl. G16H 20/60; H04W 4/02; H04W 4/021; G06Q 50/12

(52) U.S. Cl. CPC G16H 20/60; G06Q 50/12; H04W 4/021; H04W 4/023

(57) ABSTRACT

Systems and methods comprise receiving a restaurant query from a device for a user meal plan, locating one or more restaurants that are less than a threshold distance from the device, calculating a current target aggregated nutritional value for the user meal plan, and determining at least one restaurant menu item of the one or more restaurants is within the current target aggregated nutritional value. The at least one restaurant menu item and an aggregated nutritional associated with each of the at least one restaurant menu items are further displayed. The aggregated nutritional values may be B-Count values that each represent a predetermined macronutrient balance and a caloric content for a user meal plan. The one or more restaurants may be located based on detected GPS coordinates.

### CROSS REFERENCE TO RELATED APPLICATIONS

The present application is a continuation-in-part of U.S. Non-Provisional application Ser. No. 14/856,430 entitled “SYSTEMS AND METHODS FOR ACHIEVING AND MAINTAINING BEHAVIORAL FITNESS,” filed on Sep. 16, 2015, which claims priority to U.S. Provisional Patent Application No. 62/051,786, entitled “SYSTEMS AND METHODS FOR ACHIEVING AND MAINTAINING BEHAVIORAL FITNESS,” filed on Sep. 17, 2014, the entire contents of which are hereby incorporated by reference for all purposes.

### BACKGROUND/SUMMARY

Many people seek to increase physical fitness levels and/or lose weight, and a variety of programs exist to help people exercise, eat healthy meals, etc. However, even given the advanced technology increasingly being incorporated into such programs, many people still find it difficult to maintain an exercise and/or diet routine. For example, such programs may not be tailored to a given user, may lack suitable progress monitoring, or may provide the user with an unsatisfactory solitary experience.

Fitness and readiness are especially important for military reserve members who must meet physical fitness requirements and constantly be prepared for combat mobilization. However, military reserve members live as civilians and are more or less isolated from the strongly regimented routines of military life. As such, achieving and maintaining a physically fit lifestyle can be challenging without proper motivation and guidance.

Furthermore, resilience is equally as important as physical fitness, especially for military reserve members. For example, even a physically fit military reserve member may find him or herself overwhelmed by the mental strain of combat. However, physical fitness programs typically lack adequate training for mindfulness and strong, positive attitudes. As such, many people, especially military reserve members, may benefit from a behavioral fitness program that offers holistic training for physical fitness and resilience.

Further still, the inventors have recognized the importance of being able to support a behavioral fitness program in a variety of situations. For example, users following a particular meal plan may find it particularly difficult to adhere to the meal plan due to a complexity in not only a caloric target set by the meal plan, but also by a macronutrient balance set by the meal plan and, in some cases, even further requirements such as time-based requirements. Following meal plan with several requirements is made further challenging when users are traveling or otherwise in a situation where they may be eating at a restaurant.

Because restaurant menus are distributed across various locations and are in non-standardized formats, there are significant technical challenges in being able to identify one or more restaurant menu items that satisfy the current targets of a user's meal plan. Even if one or more restaurant menu items satisfying the user's current meal plan targets are able to be identified, displaying such information in a manner that conveys the macronutrient content and caloric content is a further technical challenge.

Previous approaches, such as U.S. Publication No. 2013/0216982, have simply displayed a list of multiple macronutrient contents and a caloric content of menu items in order to convey such information to a user. However, because the information in these previous approaches individually represents the macronutrients and caloric content, these previous approaches are often unable to fit on a user display in a manner that allows the macronutrient balances and the caloric content to be easily viewed. Even if these previous approaches are able to fit all of the information on a user display, there are so many considerations that it is still difficult to compare the menu items in the context of the user's meal plan (e.g., a ratio of multiple macronutrients relative to one another for each menu item, a caloric content for each menu item, a time of day, etc.).

The inventors have recognized the above issues and have devised several approaches to address them. In particular, systems and methods for adhering to a behavioral fitness routine are provided. In one embodiment, a method comprises: receiving, from a user via a user device, a health balance goal for the user; receiving, from a coach via a coach device, approval of the health balance goal; automatically generating a meal plan, a workout plan, and a mindfulness plan based on the health balance goal; receiving, from the user via the user device, data indicating one or more user activities related to one or more of the meal plan, the workout plan, and the mindfulness plan; and automatically generating a user interface for display via one or more of the user device and the coach device, the user interface visually depicting user progress towards meeting the health balance goal, the user progress based on the one or more user activities. In this way, users can establish reasonable fitness, nutrition, and wellness goals and can be held accountable to those goals by other users and stakeholders.

Further, in one or more examples, a method or system may receive a restaurant query for a user, determine at least one restaurant menu item is within a current target B-Count for the user, and display the at least one restaurant menu item and a B-Count value associated with the at least one restaurant menu item, wherein the B-Count value is a value that indicates a predetermined macronutrient balance and a caloric content. It is noted that the B-Count value disclosed herein is an aggregated value calculated based on macronutrient and caloric values. Thus, the B-Count value (which is a single aggregated value) differs from a traditional nutrition chart, as there is no single value on a nutrition chart that conveys the information conveyed by the B-Count value disclosed herein.

Via such an approach, non-standardized nutritional information from restaurant menus is advantageously converted to a standardized B-Count format, where the standardized B-Count format is concise while still conveying a substantial amount of information (e.g., conveys a macronutrient balance and a caloric content via a B-Count value). In turn, the technical effect of enabling a substantial amount of nutritional information to easily be displayed is achieved, allowing for information to be conveyed even on displays with significant size constraints and enabling an easy comparison of potential meal plan options. Further technical effects achieved include reduced computing requirements achieved via the standardized B-Count format while maintaining a substantial amount of information. For example, the standardized B-Count format of the present disclosure enables a macronutrient balance and caloric content of items to be transmitted with reduced bandwidth requirements and stored with reduced storage requirements compared to previous approaches which may transmit and store data in a format that individually represents multiple macronutrients for each item and a caloric content for each item.

Moreover, technical effects as to enabling a holistic comparison of food options is achieved via the B-Count format disclosed herein. In particular, by comparing food options in the B-Count format, the food options are able to be compared based on macronutrient and caloric content in a standardized manner.

Overall, the approach herein performs location-based restaurant menu parsing that is customized to output results highly specific to current meal plan targets of a particular user. Via the use of calculating aggregated values to achieve a standardized format, where in the standardized format a single value conveys both a predetermined macronutrient balance and caloric content personalized to a user, comparisons between current meal plan targets and restaurant menu options for the user are able to be made with high accuracy and in a location-sensitive manner.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE FIGURES

FIG. 1 shows an example computing environment.

FIG. 2 shows an example information flow.

FIG. 3 shows a high-level flow chart illustrating an example method for defining fitness goals.

FIG. 4 shows a high-level swim-lane flow chart illustrating an example method for monitoring fitness progress.

FIG. 5 shows a high-level flow chart illustrating an example method for creating a balanced meal plan.

FIG. 6 shows a high-level flow chart illustrating an example method for locating balanced meals at nearby restaurants.

FIG. 7 shows an example interface illustrating an example method for locating balanced meals at nearby restaurants.

FIG. 8 shows a high-level flow chart illustrating an example method for logging activity via text message.

FIG. 9 shows a high-level flow chart illustrating an example method for logging activity via a paper-based journal.

FIG. 10 shows an example interface for a detailed overview of weekly progress for a user and the user's team members.

FIG. 11 shows an example interface for a detailed overview of weekly progress for a user.

FIG. 12 shows an example interface for logging sleep notes.

FIG. 13 shows an example interface for displaying goals versus actual performance on a weekly basis.

FIG. 14 shows an example interface for a daily meal plan.

FIG. 15 shows an example interface for logging a meal.

FIG. 16 shows an example interface for creating a balanced meal recipe.

FIG. 17 shows an example interface for displaying weekly motivational images.

FIG. 18 shows an example interface for a leadership dashboard.

FIG. 19 shows an example interface for displaying aggregated qualitative metrics.

FIG. 20 is a schematic diagram illustrating an example network architecture for a behavioral fitness system.

FIG. 21 shows a block diagram illustrating a server system.

### DETAILED DESCRIPTION

The present description relates to systems and methods for achieving and maintaining physical fitness. In particular, systems and methods are provided for setting and tracking specific goals regarding fitness, mindfulness, sleep, and nutrition in the context of a prescribed behavioral fitness program to gain improvements in those areas and reporting on progress to stakeholders. As used herein, “stakeholders” refers to one or more coaches, team members, or other users who have an interest in the behavioral fitness of a given user. For example, a user of the systems and methods provided may be a member of a military reserve guard. As such, team members may refer to, for example, other reserve guard members with whom the user is familiar. A coach may refer to a military member with whom the user is familiar, or may refer to a person specifically trained to coach a user towards a balanced health. Other stakeholders may include military leadership, for example, military officers to whom the user may report.

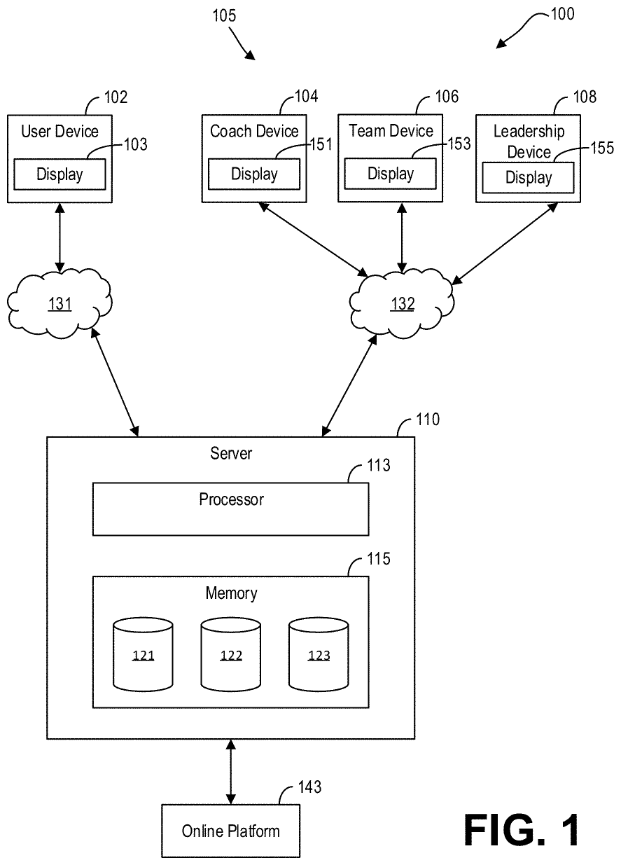

FIG. 1 shows an example computing environment 100 that can be configured to implement exemplary embodiments of the present invention. In particular, computing environment 100 illustrates the interaction between user device 102, coach device 104, team device 106, leadership device 108, and server 110.

User device 102 may comprise a computing device capable of receiving and transmitting information. User device 102 is shown in simplified form. However, user device 102 may take the form of one or more personal computers, server computers, tablet computers, home entertainment computers, network computing devices, gaming devices, mobile computing devices, mobile communication devices (for example, smart phone, smart watch, etc.), and/or other computing devices. As another example, user device 102 may comprise a cellular phone with or without functionality for connecting to a network such as the Internet. User device 102 may include a display 103.

Computing environment 100 may further include a variety of stakeholder devices 105, such as coach device 104, team device 106, and leadership device 108. Similar to user device 102, each stakeholder device 105 may comprise a computing device capable of transmitting information and is shown in simplified form. Each stakeholder device 105 may take the form of one or more personal computers, server computers, tablet computers, home entertainment computers, network computing devices, server computers, tablet computers, home entertainment computers, network computing devices, gaming devices, mobile computing devices, mobile communication devices (for example, smart phone), and/or other computing devices. Each stakeholder device 105 may include a display, such as display 151, display 153, and display 155.

Computing environment 100 may include a server 110. Server 110 may include a logic machine, such as processor 113. Processor 113 may include one or more physical devices configured to execute instructions. For example, processor 113 may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

Processor 113 may be single-core or multi-core, and the instructions executed thereon may be configured for sequential, parallel, and/or distributed processing. Individual components of the processor 113 optionally may be distributed among two or more separate devices, which may be remotely located and/or configured for coordinated processing. Aspects of the processor 113 may be virtualized and executed by remotely accessible, networked computing devices configured in a cloud-computing configuration.

Server 110 may further include a storage machine, such as memory 115, configured to hold instructions executable by processor 113 to implement the methods and processes described herein. When such methods and processes are implemented, the state of memory 115 may be transformed—for example, to hold different data. Memory 115 may include removable and/or built-in devices. Memory 115 may include optical memory (for example, CD, DVD, HD-DVD, Blu-Ray Disc, etc.), semiconductor memory (for example, RAM, EPROM, EEPROM, etc.), and/or magnetic memory (for example, hard-disk drive, floppy-disk drive, tape drive, MRAM, etc.), among others. Memory 115 may include volatile, nonvolatile, dynamic, static, read/write, read-only, random-access, sequential-access, location-addressable, file-addressable, and/or content-addressable devices.

Furthermore, server 110 may further include one or more databases in non-transitory memory 115, such as nutrition database 121, exercise database 123, and mindfulness database 125. In some examples, these databases may be designed to match military training routines and certain levels of military activity.

Server 110 may interact with user device 102 via network 131. In some examples, user device 102 may be a computing device configured with network connectivity, and so network 131 may comprise the public Internet, a private Internet, an intranet, and the like. In other examples, user device 102 may be a cellular phone with limited network functionality for connecting to, for example, the Internet, and so network 131 may comprise a cellular network. In yet other examples, user device 102 may comprise a plurality of devices with or without network connectivity, and so network 131 may comprise a plurality of networks such as the Internet and/or a cellular network.

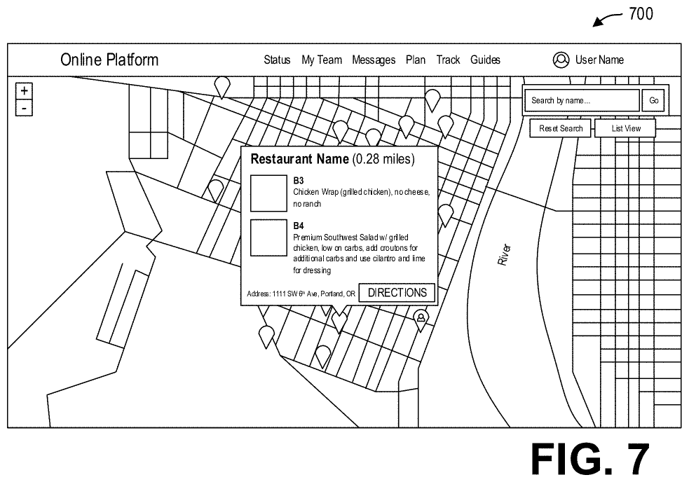

Server 110 provides an online platform 143 for tracking behavioral fitness progress. Users of user device 102 and stakeholder devices 105 may access online platform 143 in multiple ways, for example, via a website, a mobile phone application, text message, picture message, and the like. Online platform 143 provides an interface for establishing behavioral fitness targets, generating a plan to meet behavioral fitness targets, tracking progress towards behavioral fitness targets, determining the nutritional content of a meal, determining where to find balanced meals at nearby restaurants, communicating with coaches, team members, and leadership, and the like. Example interfaces for online platform 143 are described further herein and with regard to FIGS. 7 and 10-25.

In one example, computing environment 100 provides a system for remotely coaching an individual to improve mindfulness, fitness, sleep, and nutrition through accountability to system-identified stakeholders. Multiple tools may be integrated into the accountability system to facilitate setting adherence targets, capturing data, reporting on user's progress, and/or modifying the user's program to assist in achieving adherence targets.

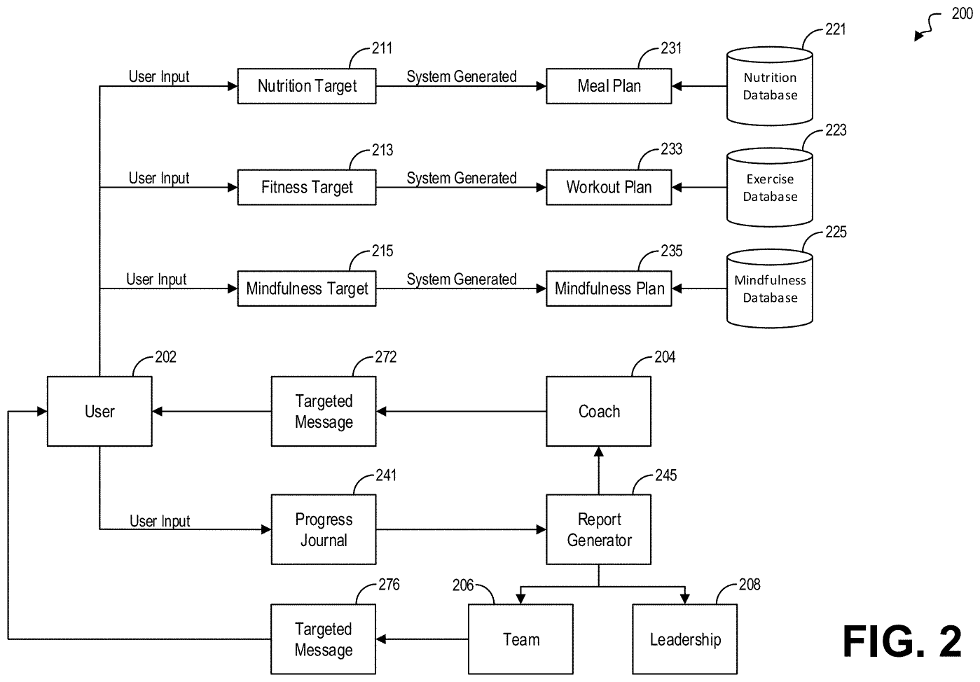

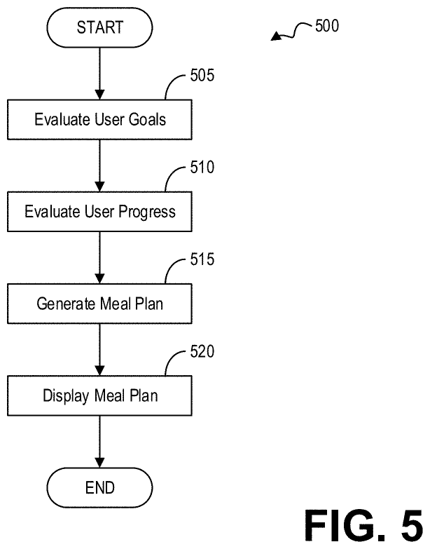

For example, server 110 may use information stored in non-transitory memory 115 to generate plans tailored to a user based on user-defined goals. For example, a user may set a nutrition target, a fitness target, and a mindfulness target. Server 110 may use these goals to generate a personalized meal plan using information from nutrition database 121, a personalized workout plan using information from exercise database 123, and a personalized mindfulness plan using information from mindfulness database 125. Generating user-specific plans based on user-defined goals is described further herein and with regard to FIGS. 2 and 5.

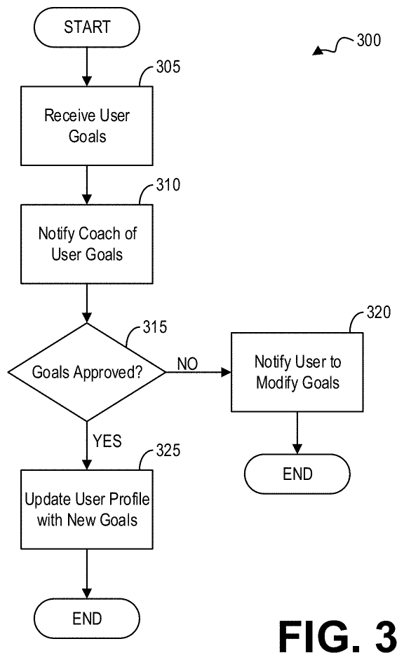

Further, the goals set by the user may be approved by a coach. A coach may be a friend, colleague, peer, and so on. In some examples, a coach may be, for example, a military veteran specifically trained with skills to effectively coach users towards meeting their goals. In this way, a user may work with an experienced coach on setting personal fitness, nutrition, and mindfulness goals. In another example, a coach may be a computerized entity. For example, server 110 may include software to emulate an experienced, human coach. A method for setting goals with approval from a coach is described further herein and with regard to FIG. 3.

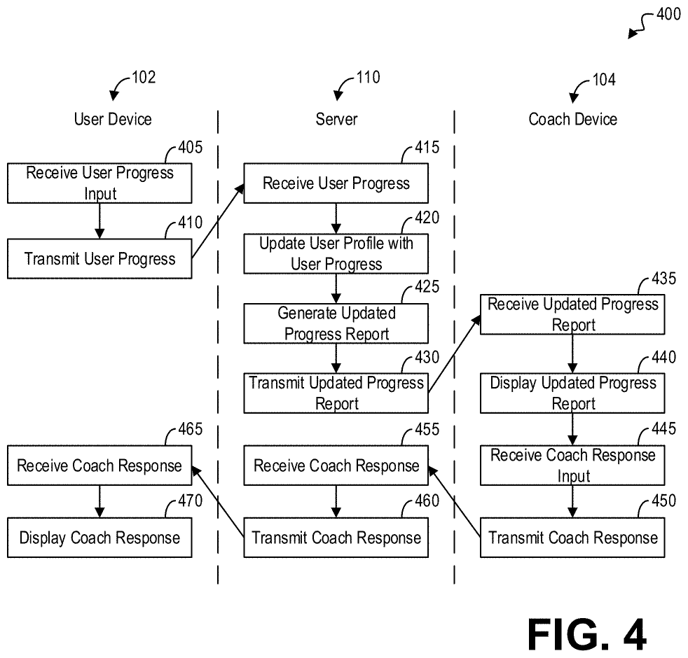

Online platform 143 may be used to log progress with respect to the plans and goals in a progress journal. Users may input activity such as exercise performed, meals consumed, number of hours of sleep, and so on via online platform 143. Online platform 143 may process the activity logged to generate a progress report which may be shared with stakeholders, such as a coach, team members, and leadership. Stakeholders may review progress reports and send targeted messages to the user in response to the progress reports. Logging activity in a progress journal, generating progress reports, and enabling communication between a user and a stakeholder is described further herein and with regard to FIGS. 2 and 4. By the online platform 143 provided by the server 110 processing the activity logged to generate the progress report, technical advantages in terms of reduced computing load on the user devices 102 and stakeholder devices 105 may be achieved. In turn, improved functioning of the user devices 102 and the stakeholder devices 105 may result while still being able to support the tracking of user progress via the user devices 102 and the stakeholder devices 105. Moreover, because the online platform 143 provided by the server 110 processes the user inputs to generate the progress reports, as opposed to the user devices 102 and the stakeholder devices 105, updates are able to be made to the system while avoiding interruption at the user devices 102 and the stakeholder devices 105.

In one example, a customized meal plan may be generated by server 110 so that a user may know what and when to eat in order to meet his or her goals. A meal plan may comprise a specified number of meals and snacks at certain times in the day, where the meals and snacks may be balanced in terms of caloric content and macronutrient content (e.g., carbohydrates, protein, fat). It is noted that the terms meals and snacks may be used interchangeably herein. In order to facilitate ease of consumption, meal plans may be presented in terms of B-Count targets. B-Counts represent a total number of calories in a balanced meal. That is, a B-Count value is assigned to a balanced meal, where the balance meal is a meal that satisfies a predetermined macronutrient balance according to the meal plan for the user. The B-Count value further indicates a caloric content of the meal. Thus, the B-Count value is a value that indicates a combination of a macronutrient content and caloric content. In this way, a simple system is provided to a user so that the user may easily identify what to eat in order to meet his or her goals and to track their daily consumption. In particular, the B-Count format disclosed herein enables presentation of a macronutrient balance and caloric content for a meal that may be time-specific depending on the meal plan in a manner that is concise. Via this concise B-Count format, technical challenges in terms of being able to present the macronutrient content and caloric content for one or more meals is achieved. The B-Count format further solves technical challenges in terms of enabling meals to be easily compared to one another while catering to the various requirements, such as macronutrient, caloric, and time-based (e.g., time of day), requirements for an individual's meal plan. B-Counts are described further herein with regard to FIG. 5.

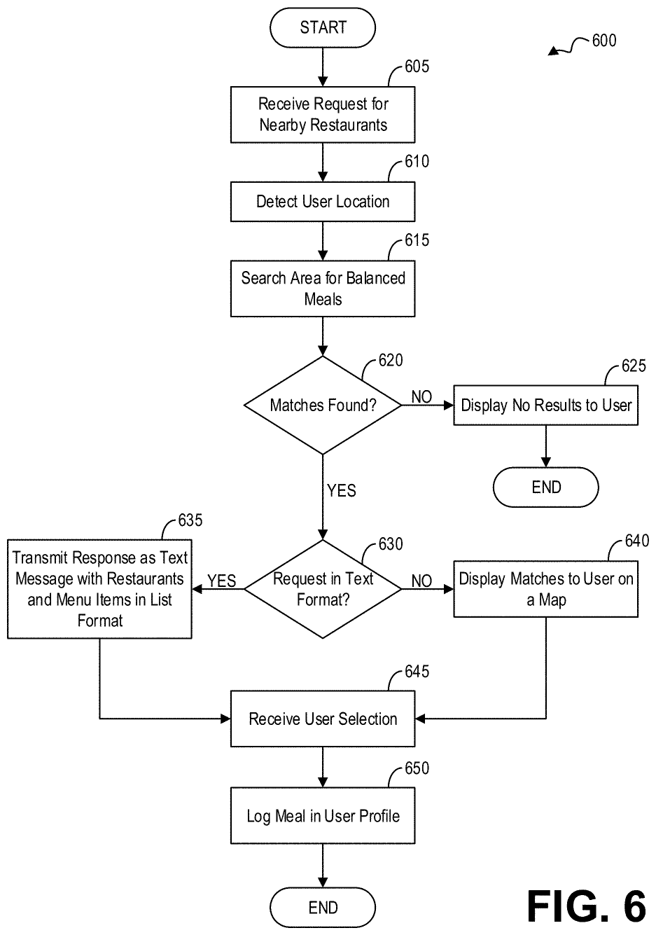

In order to facilitate adherence to a meal plan for users who may have busy or unpredictable schedules, users need an easy way to find balanced meals within target B-Counts at nearby restaurants and establishments. Hence, a geolocation feature is provided that allows users to identify nearby restaurants and establishments and view balanced meals within target B-Counts. A user may then log his or her consumption of these meals to count toward predefined nutrition targets. A method and interface for such a geolocation feature is described further herein and with regard to FIGS. 6 and 7.

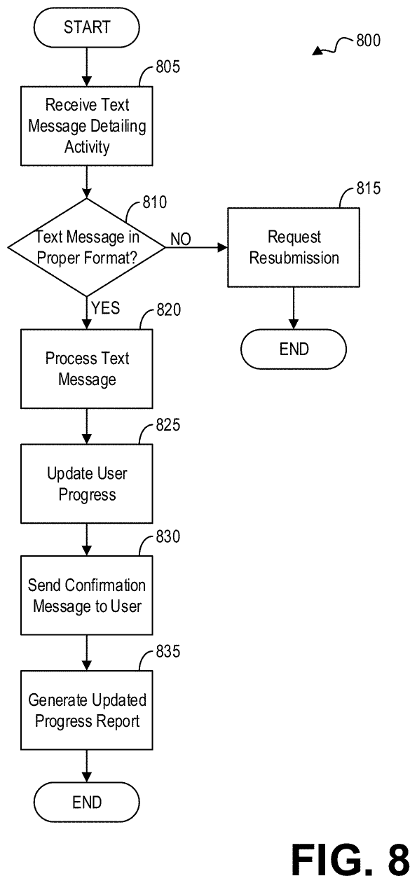

Some users may prefer not to use or may be unable to use online platform 143. For example, some users may not have a smart phone enabling simple interaction with a mobile application, or may not have regular access to the Internet. Such users may be accommodated in multiple ways. In some examples, a user may interact with online platform 143 via short message service (SMS), or a text message. For example, a user may send a text message containing a progress status to server 110 via a specified phone number. The text message may be formatted in a specific way in order for server 110 to interpret the text and update the user's profile. In these examples, user device 102 may be a cellular phone and network 131 may comprise a cellular network. A method for interacting with online platform 143 via text message is described further herein and with regard to FIG. 8.

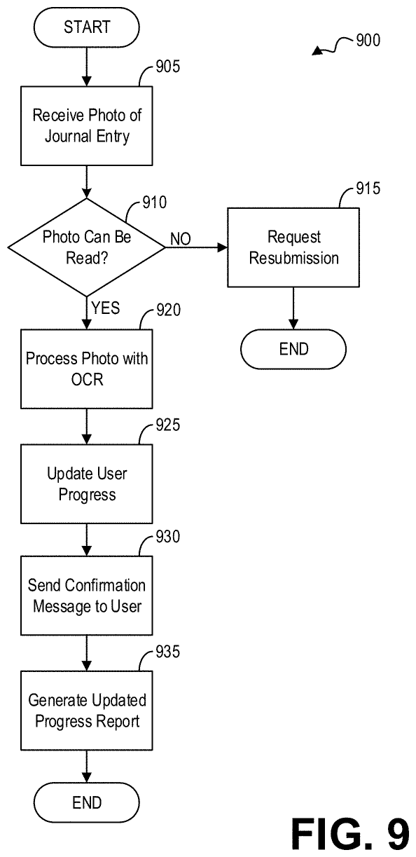

In other examples, a user may interact with online platform 143 via a photo message. For example, a user may prefer to log his or her workouts and meals in a physical journal throughout the day. The user may use a cell phone equipped with a camera to take a digital photograph of the journal page, and may send this digital photograph to server 110. The photograph may be sent as a picture message using, for example, a multimedia message service (MMS), electronic mail, and the like. Server 110 may use optical character recognition (OCR) technology to interpret the text on the journal page and update the user's profile with the interpreted text. A method for using a physical journal and OCR technology to track progress is described further herein and with regard to FIG. 9.

In some examples, a user may be grouped into a team of similar users. Such a team may comprise users who participate in the same military unit, live in the same geographical area, and so on. A team may include a single coach who coaches each member of the team and to whom each member is accountable. Furthermore, each user within a team may directly interact with other team members, for example, each user may be able to view individual and group statistics towards progress goals and communicate with one another about physical fitness and mindfulness. In this way, group cohesion may be developed while users participate in the fitness program. In some examples, each user may be assigned a team partner, or buddy, with whom they are more personally accountable. Server 110 may provide an interface that enables private communication and support between buddies, and in this way each user may be held personally accountable for him or herself as well as a buddy. In some examples, such teams may be structured similarly to groups within military units. For example, each team may comprise eight to fifteen users, similar to a military squad or section. In this way, the benefits of military organization, such as group cohesion, may be achieved.

In yet other examples, online platform 143 may provide one or more questionnaires to each user. Such questionnaires may be used to determine the physical and mental state of a user, and may be regularly administered in order to gain more in-depth knowledge regarding a user's progress towards physical and mental fitness.

FIG. 2 shows a schematic diagram illustrating an example information flow 200. Information flow 200 may represent the exchange of information between a user 202, a coach 204, a team 206, and leadership 208 via a computing environment, such as example computing environment 100. Information flow 200 will be described with regard to the systems and components of FIG. 1, though it should be understood that other systems and components may be used in accordance with the current disclosure.

User 202 inputs a variety of behavioral fitness goals, such as nutrition target 211, fitness target 213, and mindfulness target 215. Server 110 may generate customized plans for user 202 to follow in order to meet the user-input targets, such as a customized meal plan 231 for the nutrition target 211, a customized workout plan 233 for the fitness target 213, and a customized mindfulness plan 235 for the mindfulness target 215. For example, a meal plan may prescribe a number of meals with specified nutritional content, a fitness plan may prescribe a daily workout plan, and a mindfulness plan 235 may prescribe a daily meditation routine, a number of pages in specified books to read, a number of hours per night to sleep, and so on. In some examples, a mindfulness plan 235 may comprise the act of logging a user's physical activity and nutritional consumption on online platform 143, thereby increasing the user's mindfulness of their habits. In other examples, a mindfulness plan 235 may comprise a set of weekly mindfulness or resilience principles that a user may review and contemplate each week. Additionally, a user may set a resilience or mindfulness goal that corresponds to a weekly mindfulness principle, and may submit a motivational image that corresponds to the mindfulness goal. Such mindfulness images may be displayed throughout online platform 143 to the user and other users in order to serve as constant reminders of the mindfulness plan 235.

In some examples, the databases and system-generated plans may be designed to match military training routines and certain levels of military activity. For example, some aspects of fitness plans may include standard workouts implemented in basic military training. In this way, a user adhering to the system-generated plans may recall the experience of fitness obtained while actively serving in the military, and may therefore be more mentally and physically prepared for combat mobilization at a moment's notice.

As the user 202 follows the system-generated plans, user 202 tracks progress in a progress journal 241. Progress journal 241 is a log of all user activity towards the specified goals. In some examples, progress journal 241 may have a paper-based analog that a user may primarily utilize for tracking personal progress. In such examples, the user may upload photographs of the paper-based journal entries, and server 110 may, for example, use OCR technology to interpret the photographs and update progress journal 241 with the interpreted journal entry. By processing the photographs and updating the progress journal with the interpreted journal entry via the server 110, technical advantages are achieved in terms of reduced computing load at the user device.

Progress journal 241 and report generator 245 may be associated with a user profile on a social media platform, such as online platform 143. Report generator 245 utilizes information in progress journal 241 to generate progress reports. Coach 204 may review the progress reports generated by report generator 245, and may send a targeted message 272 to user 202. For example, targeted message 272 may comprise a note of encouragement, advice, reprimands, and the like. Similarly, team 206 comprising similar users may review the progress reports generated by report generator 245, and may send a targeted message 276 to user 202. Further, in some examples leadership 208 may review the progress reports and may also send a targeted message (not shown) to user 202. In this way, the progress of user 202 may be reviewed and commented on by interested parties. This system of review and communication may increase accountability for user 202, and serve as encouragement to follow the plans and therefore meet the targets. Furthermore, this system may comprise a social media platform that encourages open communication between all parties involved regarding progress and activity.

FIG. 3 shows a high-level flow chart illustrating an example method 300 for establishing goals with the approval of a coach. Method 300 will be described with reference to the systems and components of FIGS. 1 and 2, however the method may be applied to other systems and components without departing from the scope of this disclosure. Method 300 may be implemented as instructions in non-transitory memory on server 110.

Method 300 may begin at 305. At 305, method 300 may include receiving user goals. User goals may be input by a user 202 on user device 102 and sent to server 110 via network 131. User goals may include, as general examples, target goals regarding fitness, nutrition, and mindfulness. Particular examples of user goals may include a desired body weight, workout targets (for example, running five kilometers in less than half an hour, bench pressing 110% of the user's body weight, etc.), an amount of nightly sleep, and so on. Method 300 may then proceed to 310.

At 310, method 300 may include notifying a coach 204 of user goals. Notifying a coach 204 of user goals may comprise, for example, sending a message to coach device 104 indicating that user 202 has chosen goals. Coach 204 may review the user goals on coach device 104 and determine if the goals are appropriate for user 202. Determining if the goals are appropriate for user 202 may include considering the goals given the details of the user 202. For example, if the user currently weighs 300 pounds and chooses a goal of losing 100 pounds in one month, the coach may recognize that the goal is unfeasible given the circumstances. As another example, if the user sets a goal of doing strength training workouts for three hours a day every day of the week, the coach may recognize that such a workout schedule may be unreasonable in light of the user having a full-time job and a family life, which may be details included in a profile set up by the user. As another example, if a user submits a sleep target that includes a very large or very small number of hours of sleep per night, the coach may recognize that such sleeping habits are unhealthy.

Continuing at 315, method 300 may include determining if the user goals are approved by the coach 204. If the user goals are not approved, method 300 may proceed to 320. At 320, method 300 may include notifying user 202 to modify his or her goals in order to obtain approval from coach 204. In some examples, notifying user 202 to modify goals may include a targeted message from coach 204 indicating why the goals were not approved and/or how to modify the goals. Considering the examples discussed above, the coach may, for example, suggest a more feasible weight loss plan, or may recommend, for example, an intense workout routine that covers all aspects of physical fitness and may be accomplished in less time per day. As another example, the coach may suggest a number of hours to sleep each night that may benefit the user's mental and physical health. Method 300 may then end. After method 300 ends and the user reviews the message from coach 204, method 300 may begin again when user 202 submits new goals based on the message from coach 204. In this way, the user 202 and coach 204 may establish personally-tailored goals for user 202 by communicating with each other.

Returning to 315, if the user goals are approved, method 300 may proceed to 325. At 325, method 300 may include updating the user profile for user 202 with the new personalized goals. In some examples, method 300 may further include generating new customized plans for the user goals. Method 300 may then end.

FIG. 4 shows a high-level swim-lane flow chart illustrating an example method 400 for tracking user progress. In particular, method 400 illustrates how server 110 interacts with user device 102 and coach device 104 to track user progress and provide targeted messages from coach device 104 to user device 102. Method 400 includes coach device 104, however in some examples the steps associated with coach device 104 may also be applied to another stakeholder device, such as team device 106 and/or leadership device 108.

Method 400 may begin at 405. At 405, user device 102 may receive user progress input from a user 202. User progress may be input in multiple ways. In examples where user device 102 is a mobile computing device (e.g., a smart phone), user progress may be input as text in a mobile device application. In examples where user device 102 is a personal computer or laptop, user progress may be input as text in an interface displayed via an internet browser. In examples where user device 102 is a cellular phone, user progress may be input as text in a text message format or as a digital photograph of a paper-based journal.

After user device 102 receives the user progress input, method 400 may include transmitting the user progress from user device 102 to server 110. Transmitting user progress from user device 102 to server 110 may occur over a network 131. Network 131 may comprise a public Internet if user device 102 is capable of accessing the Internet, or network 131 may comprise a cellular network if user device 102 is a cellular device.

At 415, server 110 may receive the user progress transmitted from user device 102. In some examples, method 400 may further include processing the received user progress depending on the transmission format. For example, if the user progress is received in the form of a digital photograph, server 110 may use OCR technology to process the photograph and interpret any text detected in the photograph. As another example, if the user progress is received in the form of a text message, server 110 may process the text message and interpret the text.

Continuing to 420, server 110 may update a user profile with the received user progress. The user profile may be associated with the user 202 of user device 102. Method 400 may then proceed to 425.

At 425, server 110 may generate an updated progress report based on the user progress. The progress report may visually or textually depict the overall user progress towards the user goals.

After server 110 generates an updated progress report, method 400 may continue to 430. At 430, server 110 may transmit the updated progress report to a stakeholder device, such as coach device 104.

At 435, coach device 104 receives the updated progress report. At 440, coach device 104 displays the updated progress report so that the coach 204 may review the progress of user 202. After reviewing the progress report, coach 204 may choose to send a targeted message 272 to user 202. Targeted message 272 may comprise, for example, a note of encouragement, advice for how to improve, reprimands for not satisfying expectations, and so on.

If coach 204 chooses to send a targeted message 272, method 400 may continue to 445. At 445, method 400 may include receiving coach response input. Continuing at 450, coach device 104 may transmit the coach response to server 110.

At 455, server 110 may receive the coach response. In some examples, server 110 may store the coach response in non-transitory memory for later retrieval. At 460, server 110 may transmit the coach response to user device 102.

At 465, user device 102 may receive the coach response, and at 470, user device may display the coach response to user 202.

In this way, user 202 may be accountable for his or her progress and may be motivated to follow a fitness plan and therefore meet the established goals.

FIG. 5 shows a high-level flow chart illustrating an example method 500 for generating a meal plan appropriate for the nutritional goals and overall progress of a user. In particular, method 500 relates to the creation of balanced meals and the assignment of a caloric range based on circumstance and physical attributes of a user for each of six meals during three time periods during the day. B-Counts may represent the total amount of calories in each of the three time periods, and are described further herein. Method 500 may be stored as executable instructions in non-transitory memory on server 110.

Method 500 may begin at 505. At 505, method 500 may include evaluating user goals. Evaluating user goals may include evaluating targets such as nutrition targets, fitness targets, and mindfulness targets. For example, a user goal may include reaching a particular combination of body weight, body fat percentage, and/or body mass index. Evaluating user goals may further include evaluating the system-generated plans for achieving targets, such as a previously-generated meal plan, workout plan, and mindfulness plan. The meal plan may be personalized to the user and comprise predetermined macronutrient balance targets, caloric targets. Further, in at least one example, the meal plan may be time-based. Method 500 may then continue to 510.

At 510, method 500 may include evaluating user progress. Evaluating user progress may comprise evaluating the actual progress of the user, including actual nutritional intake and workouts. Evaluating actual progress of the user enables server 110 to determine a meal plan appropriate to the user's goals and progress.

At 515, method 500 may include generating a personalized meal plan. Generating a personalized meal plan may comprise determining daily caloric intake based on the user goals and progress. For example, prescribed caloric intake may be increased on days where the user exercises more and burns more calories, or decreased on days where the user does not have a rigorous workout routine. Moreover, the predetermined macronutrient balance for the user's meal plan may be modified depending on a user's exercise. For example, within less than a threshold period of time following an exercise routine, the macronutrient balance may be altered for a user's meal plan to require a higher percentage of protein compared to a baseline macronutrient balance for the user's meal plan. To generate a meal plan, a twenty-four hour period may be divided into three sections: morning, afternoon, and evening. The meal plan may include a meal and a snack assigned to each section, where the meal and the snack may be consumed, for example, no more than three hours apart. In at least one example, the predetermined macronutrient balance may be different at different times of day. For example, a macronutrient balance with higher carbohydrate requirements may be required at breakfast than at dinner for a user's meal plan. As such, the meal plan may have time-specific requirements.

Each meal and/or snack may be balanced according to the percentage of calories from protein, carbohydrates, fat, and macronutrient level. For example, each meal or snack may be balanced according to the percentage of calories from protein (30-40%), carbohydrates (40-50%), fat (15-25%) and level of macronutrients. In at least one example, only meals that are balanced are assigned a B-Count. That is, only meals that satisfy a predetermined ratio of macronutrient ranges may be assigned a B-Count. Thus, in such examples, all meals assigned a B-Count for a user's meal plan satisfy the predetermined macronutrient balance requirements of the user's meal plan. It is noted that since a user's meal plan may have time-specific requirements, including a predetermined macronutrient balance that varies based on a time of day or based on other conditions such as a user activity level, meals that may be assigned a B-Count at a first time may not be assigned a B-Count at a second time.

Further, as assigning a B-count to a food item indicates a predetermined macronutrient balance is achieved, and as the predetermined macronutrient balance is specific to a user meal plan, the B-count is a personalized value. Food items that receive B-counts may be different for a first user meal plan than a second user meal plan. Therefore, the B-count is both a time-based and a personalized value.

In some examples, caloric intake may be described in terms of a B-Count, where a B-Count corresponds to a total number of calories in each of the three time sections. The B-Count may be represented by an alphanumeric value. For example, the B-Count may be represented by a value that includes a letter to indicate a macronutrient balance of the meal (e.g., B) and a B number to indicate a caloric content of the meal. That is, a balanced meal may be assigned a B number according to the range of its total calories. In some examples, B numbers may defined such that B0 corresponds to zero calories, B1 corresponds to 1-100 calories, B2 corresponds to 101-200 calories, and so on. In other examples, B numbers may be defined on a different calorie scale. The total B-Count for the consumed meals may be plus or minus one of the target B-Count assigned to the user. In this way, a user may easily know what to eat and drink and at what time in order to lose weight, as the user only needs to track the B-Count rather than the specific nutrition content for each meal or snack. Moreover, due to the B-Count format, the technical effect of conveying such macronutrient balance and caloric content information that is time-based in a manner that is concise and able to be easily displayed within the physical constraints of a device display are achieved. Thus, generating a meal plan may comprise assigning a B-Count target for each time section based on the biometrics and physical activity of the user.

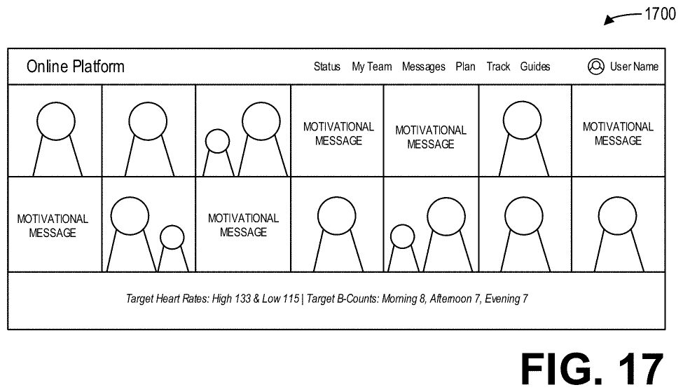

At 520, method 500 may include displaying the personalized meal plan. Displaying the meal plan is discussed further herein and with regard to FIG. 17. Method 500 may then end.

Thus, a system and method for identifying what to eat and when to eat is provided. In particular, the B-Count system described hereinabove provides a simplified way to easily identify the nutritional content of a balanced meal without needing to know the specific details of the meal, and therefore enables simple tracking of consumption.

FIG. 6 shows a high-level flow chart illustrating an example method 600 for identifying balanced meals appropriate for a nutrition plan while a user is on the go, e.g., away from home.

In at least one example, the user may be interested in dining at a restaurant while on the go. Thus, method 600 may create a geolocation restaurant guide that automatically presents location-based restaurant menu items that are customized to a user's meal plan, beginning at step 605. At 605, method 600 may include receiving a request from a user for nearby restaurants. Method 600 enables users to easily identify or modify meals at restaurants and establishments in their area while on the go.

At 610, method 600 may include detecting the location of the user. In one example, detecting the location of the user may comprise obtaining GPS coordinates of user device 102. In at least one example, the GPS coordinates may included as part of the request from the user for nearby restaurants or inferred from information in a data packet of the request received from the user for nearby restaurants. Additionally or alternatively, the GPS coordinates may be separately transmitted from the user device. The GPS coordinates may be detected based on one or more of satellite signals, Wi-Fi detected within a threshold distance of the user device, mobile networks, and dedicated GPS sensors included in the user device. In another example, detecting the location of the user may comprise receiving a location input by the user. For example, the user may include his or her location in the request at 605.

At 615, method 600 may include searching the area of the user location for balanced meals. In one example, searching the area of the user location for balanced meals may comprise searching a pre-determined list of restaurants stored on server 110. In another example, searching the area of the user location for balanced meals may comprise identifying local restaurants with online menus and analyzing the menus to determine if a menu item within a target B-Count is offered. It is noted that the target B-Count is a current nutritional target for the user, where the current nutritional target is represented by an aggregated value (e.g., a B-Count value). This aggregated value may be calculated using macronutrient meal plan targets and caloric meal plan targets for the user. Additionally or alternatively, the aggregated value for the current target B-Count may be calculated based on one or more of a food consumption tracked for the user, a time of day, exercise recorded for the user, and any one or more of the other B-Count considerations discussed herein.

Restaurants use various non-standardized restaurant menu formats that are not in B-Count format. For example, restaurant menus may use a list format that describes a menu item in text. In some examples, restaurants may provide tables with nutritional information for one or more of their menu items. Thus, analyzing the menus to determine if a menu item is within a target B-Count at step 615 includes converting the menus from the non-standardized restaurant menu formats to a standardized B-Count format. Modifying the restaurant menus so that the restaurant menus are converted to the B-Count format enables the menu items of the restaurant menus to be compared to the current target B-Count in a same standardized format.

In at least one example, converting the menus to the standardized B-Count format at step 615 may include extracting nutritional information for one or more menu items of the menus. If the restaurant menu items are not listed with their respective nutritional information, then converting the menus to the standardized B-Count format may include querying a nutritional database to estimate the nutritional content (including the caloric and/or macronutrient content) of the menu items and assign B-Counts accordingly.

In one or more examples, converting the menus to the standardized B-Count may further include determining B-Counts for modified menu items. For example, such modifications may include requests that can be made upon ordering of the food for how the food is to be prepared. Such modifications may be requests as to how the food is cooked (e.g, grilled). Additionally or alternatively, such modifications may be requesting certain menu ingredients to be withheld, such as requesting no cheese and no dressing, for example. In at least one example, such modifications may include requesting a substitute item, such as requesting a low-carb bun in place of a standard bun. Further, in at least one example, the modifications may include requests from pre-defined offerings for the menu item. For example, if ordering a salad and the restaurant offers a list of dressing options for the salad, the modifications may include which dressing to choose for the menu item. Furthermore, in one or more examples, such modifications may include requests for additions, such as add croutons.

In one or more examples, contextual inferences may be made to calculate B-Counts for modified menu items. For example, restaurants may not explicitly list the macronutrients and caloric content of the cheese included in a menu item. Thus, the approach herein may contextually infer the macronturient and caloric content of the cheese of a menu item. For example, if the menu item is a Caesar salad that includes cheese, it may be contextually inferred that the type of cheese is parmesan cheese and an estimate may be made as to an amount of the parmesan cheese that is likely used. The inference may be made based on a database (e.g., the nutritional database discussed above) that includes one or more example Caesar salads with information as to the type of cheese and the nutritional information of the cheese included in these Caesar salads. As another example, if a restaurant is an Italian restaurant and menu items include cheese, then it may be contextually inferred that the menu items utilize Italian cheeses (e.g., mozzarella). The nutritional information and serving size information available on the restaurant menu for the menu item (e.g., Caesar salad) may additionally or alternatively be used to make such a contextual inference. As another example, if predicting a caloric and macronutrient impact of ordering a chicken breast deep fried compared to ordering the chicken breast grilled, a database including examples of deep fried and grilled chicken breasts (e.g., the nutritional database discussed above) may be consulted to estimate the macronutrient and caloric content differences. Similar contextual inferences may be made for any of the menu item modifications discussed herein.

In one or more examples, determination as to which menu items to convert to the standardized B-Count format may be based in part on a current time. For example, as previously discussed herein, the macronutrient balance requirements of a user's meal plan may vary depending on the time of day. Thus, the B-Counts assigned to the restaurant menu items during the conversion to B-Count format may be time-based B-counts determined for the menu items. These time-based B-Counts may then be compared to the current target B-count to determine if the menu items are within the current target B-count.

Further, if one or more of the locally identified restaurants are closed due to the time of day, the menus for such closed restaurants may not be converted. Additionally or alternatively, if one or more of the locally identified restaurants are currently closed but are opening in less than a threshold period of time (e.g., 120 minutes), then the menus for such restaurants that are opening in less than the threshold period of time may still be converted to the standardized B-Count format. It is also possible for the restaurant menus to be converted regardless of whether the restaurants are opened or closed, so that a user may plan a meal for a potential future visit. In cases where a restaurant menu for a closed restaurant is converted to the standardized B-Count format, results from such a restaurant may be displayed with a warning that the restaurant is currently closed.

It is noted that many restaurants only serve a subset of their menu items depending on the time of day. The subset of the menu items may include one or more of a breakfast subset, a lunch subset, and dinner subset, for example. Thus, even if a restaurant is open at the current time of day when the user request for the nearby restaurants is received, not all of the restaurant menu items listed on the restaurant menu may currently be available for purchase. The current time of day may be detected via the user device or via a server (e.g., server 110) processing the user request for the nearby restaurants.

As such, in at least one example, analyzing the restaurant menus at step 615 for converting the menu items to the standardized B-Count format may further include only converting menu items that are currently being served and/or that are being served in less than a threshold period of time (e.g., less than 120 minutes) based on the current time of day.

For example, if a breakfast subset of the menu items is served at a restaurant from 8:00 am to 11:00 am, a lunch subset of the menu items is served at the restaurant from 11:00 am to 4:00 pm, and a dinner subset of the menu items is served at the restaurant from 4:00 pm to 9:00 pm, the current time of day when the user requests the nearby restaurants may impact which menu items are converted to the standardized B-Count. For example, if the user request for nearby restaurants is received at 10:30 am for a restaurant with subset menus having the above time ranges, converting the menu items may include only converting the breakfast subset of the menu items to the standardized B-Count format. Additionally or alternatively, the lunch subset of the menu items may be converted to the standardized B-Count format.

The nutritional information for the menu items is used to calculate a B-Count for each of the one or more menu items. As explained previously, a B-Count value may only be assigned to items that satisfy a predetermined nutritional balance in at least one example. Thus, it is possible that one or more of the menu items being analyzed to not be assigned a B-Count value if they do not satisfy the predetermined macronutrient balance required for the nutritional program of the user.

At 620, method 600 may include determining if a match is found. A match is determined to be found if there are one or more menu items that are within the current target B-Count. It is noted that a restaurant menu item may be also be referred to herein as a restaurant order. Forming a match may include combining one or more restaurant menu options (e.g., dishes) to create a restaurant order within the current target B-Count. For example, a restaurant order that forms a match may include multiple tacos, where the multiple tacos are the same type of taco. As another example, a restaurant order that may form a match may include a cheeseburger and a salad with no dressing. The one or more restaurant menu items, also referred to herein as restaurant orders, determined to form a match may include one or more of the modifications discussed above. In this way, restaurant menus are automatically parsed to present menu options that are highly customized and personalized to a user's meal plan while being location-based. If no match is found, method 600 may proceed to 625. At 625, method 600 may include displaying no results to the user. In some examples, method 600 may further include displaying alternative options such as nearby grocery stores, or advice for independently identifying a balanced menu item. Method 600 may then end.

Returning to 620, if a match is found, method 600 may continue to 630. At 630, method 600 may include determining if the initial user request was in a text message format. Note that method 600 refers to a text message, however any message format may be utilized. For example, the user request may take the form of a multimedia message. If the request was in text message format, method 600 may continue to 635. At 635, method 600 may include transmitting a response as a text message to the user, where the response comprises restaurant locations and menu items in list format. The menu items are menu items that are within the current target B-Count and may be sent with their respective B-Counts. If the menu items satisfying the current target B-Count require modifications, the menu items are sent and displayed with the modifications. Additionally or alternatively, if one or more of the menu items satisfying the current target B-Count require a combination of multiple menu options, then each of these one or more menu items may be sent and displayed with the multiple menu options. If the user chooses to consume one of the proposed menu items, the user may send a text message response indicating the consumed menu item.

Returning to 630, if the request is not in text message format, method 600 may continue to 640. At 640, method 600 may include displaying matches to the user on a map within the mobile application on a display of a user device to form an interactive geolocation restaurant guide that is fully customized and personalized to a user meal plan. As with the text message format, the menu items that are matches are menu items within the current target B-Count. These menu items may be sent with their respective B-Counts and displayed with their respective B-Counts on the display of the user device. A drop pin or other location indication may be provided as an overlay on the map for each restaurant displayed on the map, and a drop pin or other location indication may further be provided as an overlay on the map for a location of the user. By displaying both the restaurant and user locations, a user may easily select which restaurants are of interest and physically locate the restaurants. The user may browse the restaurants displayed on the map, for example by selecting the restaurant name or location, and view the balanced menu items. If modifications are required for a menu item to be within the respective B-Count displayed for the menu item, such required modifications are displayed with the menu item. Additionally or alternatively, if one or more of the menu items satisfying the current target B-Count require a combination of multiple menu options, then each of these one or more menu items may be sent and displayed with the multiple menu options. An example interface for displaying local restaurants that offer balanced menu items is described further herein with regard to FIG. 7. As seen at FIG. 7, a restaurant with two menu items satisfying the current target B-Count is shown. A first menu item has a respective B-Count of B3 and is a menu item that requires modifications. In particular, the example at FIG. 7 shows a B3 B-Count menu item that requires a Chicken Wrap to be ordered with the modifications of grilled chicken, no cheese, and no ranch. At the same restaurant in the example at FIG. 7, a second menu item satisfying the current target B-Count is shown. The second menu item has a respective B-Count of B4 and is also a menu item that requires modifications. In particular, the B4 B-Count menu item requires a Premium Southwest Salad to be ordered with the modifications of grilled chicken, the addition of croutons, and using cilantro and lime for the dressing. Notably, the B4 example at FIG. 7 also provides an explanation for the requirement of modifying the B4 order with the addition of croutons. In particular, the B4 menu item is explained to be low on carbs and then a modification of adding croutons for additional carbs is instructed. In this way, by providing an explanation for menu modifications, a user is educated as to why a modification is being requested.

As can be seen in the example at FIG. 7, the B-Count format conveys an indication of the balance of macronutrients and caloric content that may be time-based in a concise manner that is able to easily fit into even small user device displays (e.g., a mobile communication device). Furthermore, the B-Count format further enables the nutritional information of menu items to be more efficiently be compared to one another, while still accounting for various meal plan requirements, including a required balance of macronutrients and caloric content. If the user chooses to dine at a particular restaurant, the user may select the menu item within the application to indicate that the user will consume the menu item. Upon such selection, it is note that the current target B-Count will automatically be updated. In at least one example, menu items that were within the current target B-Count prior to the selection of the menu item may be compared to the updated current target B-Count following the selection of the menu item and removed from the display if they no longer are within the current target B-Count.

Whether the user browsed restaurant options and selected a menu item via text message at 635 or on a mobile application at 640, method 600 continues to 645. At 645, method 600 may include receiving the user selection. In examples where the user selection is received via text message, method 600 may further include logic to properly process the user selection. Such logic is described further herein with regard to FIG. 8.

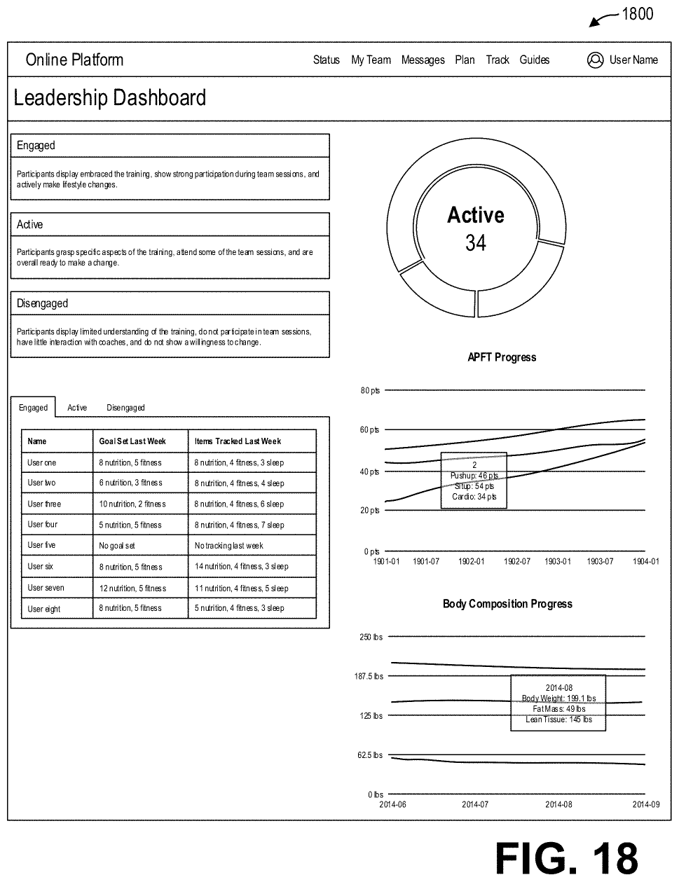

At 650, method 600 may include logging the selected meal in the user profile, as described further herein with regard to FIG. 18. Method 600 may then end.

FIG. 7 shows an example interface 700 illustrating an example method for locating balanced meals at nearby restaurants. In particular, interface 700 displays a geolocation restaurant guide. Interface 700 may comprise the interface displayed to a user at step 640 in method 600, for example. Interface 700 may include a marker indicating a user's location, in addition to markers indicating the location of restaurants that offer balanced meals. A user can click on one of the restaurant markers to view more detailed information. As shown, interface 700 may display a list of menu options listed by B-Count, as well as location information such as an address and distance from the user's current location. Interface 700 may further offer directions to the restaurant upon the user's request.

Several technical advantages are achieved by utilizing the unconventional standardized B-count format as disclosed for restaurant and meal plan nutritional information. For example, the standardized B-count format improves system operation, as the system is able to accurately and efficiently identify menu items that satisfy meal plans with multiple nutritional requirements for a particular time context (e.g., particular macronutrient and caloric content requirements). Specifically, due to the time-based B-counts of the standardized B-count format, the disclosed approach enables systems to operate accurately and efficiently while accommodating menu plans developed with high levels of specificity.

Since a time-based B-count according to the standardized B-count format represents a combination of macronutrient content and caloric content, the identified menu items that are within the current target B-count satisfy the meal plan requirements from both a macronutrient standpoint and a caloric standpoint in the context of a particular time of day. Moreover, due to the standardized B-count format, the system is able to quickly analyze the menu items of restaurant menus in B-count form to determine if they meet macronutrient and caloric meal plan requirements represented by the current target B-count for a particular time of day.

In contrast, previous approaches instead process restaurant and meal plan nutritional information without a standardized value to represent a combination of macronutrients and caloric content with meal plan nutritional constraints for a particular time of day. These previous approaches are at a great disadvantage compared to the system disclosed herein. This is not least because such previous approaches must individually calculate each nutritional requirement of a meal plan (e.g., each macronutrient requirement and caloric requirement) and then individually compare each nutritional requirement of the meal plan to corresponding restaurant nutritional information of each menu item to provide menu items that satisfy a current target macronutrient balance and a caloric content for a meal plan. The conversion to B-Count format for comparison purposes have significant computing efficiency advantages in this regard.

Due to the numerous calculations and comparisons that must be made, these previous approaches have higher computing requirements and are inefficient compared to the system utilizing the standardized B-count format disclosed herein. In turn, a processing time for these previous approaches is much longer than the disclosed approach to provide menu items within current macronutrient balance and caloric content requirements for a meal plan.

Further, even performing these calculations and comparisons, such previous approaches are not time-based and thus fail to provide results that are as customized as in the disclosed approach.

Moreover, by utilizing the standardized B-Count format of the disclosed approach that assigns time-based B-Counts as discussed above as part of an updated user interface, significant advantages are achieved as to a graphical user interface (GUI). In particular, via a single time-based B-Count value, the disclosed approach is able to indicate that a particular balance of macronutrients for a particular meal plan is satisfied, provide an indication of a caloric content, and have these results customized to the time of day based on the meal plan. In other words, the GUI is customized and standardized based on the time of the request. Thus, the time-based B-Count value of the disclosed system is able to convey a substantial amount of information with a single value, and this single value is further both personalized in a nutritional and time-specific manner to a user's meal plan.

In view of the above, the standardized B-Count format advantageously enables GUIs that are compact and easily readable without compromising on the amount of information conveyed. This advantage is particularly important in a case where a client system may be a mobile device with a relatively small screen size, for example.

In contrast, to convey the nutritional information provided by way of the time-based B-Count, previous approaches (such as those discussed above) would have to list out individual macronutrient ranges and caloric contents, which requires a substantial amount of room and/or is not easily readable on a GUI. Moreover, even in doing so, such nutritional information would still not be time-based.

Further, transmitting nutritional information represented in the standardized B-Count format reduces a file size compared to previous approaches that send files containing nutritional information for the individual macronutrients and caloric content. Thus, a transmission speed of the disclosed system to provide restaurant menu items that satisfy macronutrient and caloric content requirements is faster compared to the previous approaches. Moreover, due to the unconventional standardized B-Count format utilizing time-based B-Counts, such transmission speeds are faster compared to the prior art even though more information is being conveyed. For similar reasons, storing a meal plan in the B-Count format advantageously requires less storage space than the meal plans of such previous approaches even though more information is being stored.

In addition to the above advantages, the standardized B-Count format in the approach developed greatly improves a user experience. Specifically, by representing meal options and a user meal plan in the standardized B-Count format, a user is able to easily compare menu options to their meal plan macronutrient and caloric requirements while also satisfying meal plan time requirements. In particular, users are able to easily and quickly identify menu items that satisfy the macronutrient requirements of their meal plan and have an indication as to the caloric content of the menu items while satisfying time requirements of the meal plan.

In contrast, previous approaches that provide the individual macronutrient and caloric content information for the restaurant menu items make it difficult for a user to compare menu options to their meal plan requirements. This is not least because a user must individually compare macronutrient ranges and a caloric content of each menu option to the corresponding macronutrient and caloric content requirements for their meal plan.

Therefore, in view of at least the above, the B-Counts as utilized in the disclosed approach achieve significant technical and user experience advantages over previous systems.

FIG. 8 shows a high-level flow chart illustrating an example method 800 for updating progress via text message. Not all users will have access to the online platform or a smartphone. Therefore, users need a way to update their progress in near real-time without access to the Internet. Text messages allow participants to track and log their fitness, nutrition, and mindfulness activities to be captured by the online platform. As described herein, user data sent via text may be parsed by the system and automatically uploaded to the database for graphic display and reporting. While method 800 relates to the use of text messages, it should be appreciated that method 800 may be applied to other messaging formats, such as picture messages, e-mails, physical mail, and so on.

Method 800 may begin at 805. At 805, method 800 may include receiving a text message detailing user activity.

At 810, method 800 may include determining if the text message is in a proper format. A proper format for the text message may comprise, for example, a specified syntax to aid an interpretation of the message by server 110. A proper format may further comprise a sufficient amount of detail. As an illustrative and non-limiting example, a properly formatted text message detailing a cardio workout comprising jogging three miles in 27 minutes beginning at 6:30 P.M. may read “Jogging; 3 miles; 27 min; 18:30” while an improperly formatted text message may read “ran 3 miles in 27.” If the text message is not in the proper format, method 800 may continue to 815. At 815, method 800 may include requesting resubmission. Requesting resubmission may comprise sending a text message to the user indicating that the text message received was improperly formatted, and request to resubmit the activity update in the proper format. Method 800 may then end.

Returning to 810, if the text message is in the proper format, method 800 may continue to 820. At 820, method 800 may include processing the text message.

At 825, method 800 may include updating the user progress. After the user profile is successfully updated with the user progress, method 800 may proceed to 830.

At 830, method 800 may include sending a confirmation message to the user.

At 835, method 800 may include generating an updated progress report based on the updated user progress. Method 800 may then end.

Thus, a method and apparatus are provided for users to track and log progress in online platform 143 via text message. In this way, users may text progress toward fitness, nutrition, sleep, and mindfulness activities to be auto-populated into their online profile.

FIG. 9 shows a high-level flow chart illustrating an example method 900 for updating a user's progress with a photo of the user's journal. Not all participants will have access to the web platform or want to use the platform to track their progress. Participants who would prefer to track their progress in a physical journal need a way to update their progress on the platform. Optical character recognition (OCR) allows participants to track and log their fitness, nutrition, and mindfulness activities in a physical journal to be uploaded into the platform. A photo uploaded by participants will be parsed by the system, uploaded, and automatically inserted into the database for graphic display and reporting.

Method 900 may begin at 905. At 905, method 900 may include receiving a digital photograph of a journal entry.

At 910, method 900 may include determining if the photo can be read. In one example, the photograph may be too blurry for OCR software to correctly process the image. In another example, the photograph may be clear but the handwriting may be illegible. Thus, determining if the photo can be read may comprise determining if any handwriting identified in the photograph is, for example, substantially blurred. If the photo cannot be read, method 900 may continue to 915.

At 915, method 900 may include requesting resubmission. Requesting resubmission may comprise sending a message to the user indicating that server 110 could not process the photograph, and requesting the user either submit another photograph of the journal entry or submit a photograph of a rewritten journal entry. Method 900 may then end.

Returning to 910, if the photo can be read, method 900 may continue to 920. At 920, method 900 may include processing the photo with optical character recognition software to detect any text in the journal entry. The user activity detailed in the journal entry may be interpreted from the detected text.

At 925, method 900 may include updating the user progress.

At 930, method 900 may include sending a confirmation message to the user.

At 935, method 900 may include generating an updated progress report. Method 900 may then end.

Hence, a method and apparatus is provided for users to upload their progress tracked in a physical journal into the online platform 143. Optical character recognition technology enables users to take a picture of their physical entries of progress toward fitness, nutrition, and mindfulness activities to be auto-populated into their online profile.

When setting up an account, a sign-up interface may enable a user to enter personal information such as name, contact information, age, gender, typical activity level, height, weight, and the like. The sign-up interface may further include input fields to provide additional details regarding the user, for example, work schedule, typical daily routine, and so on.

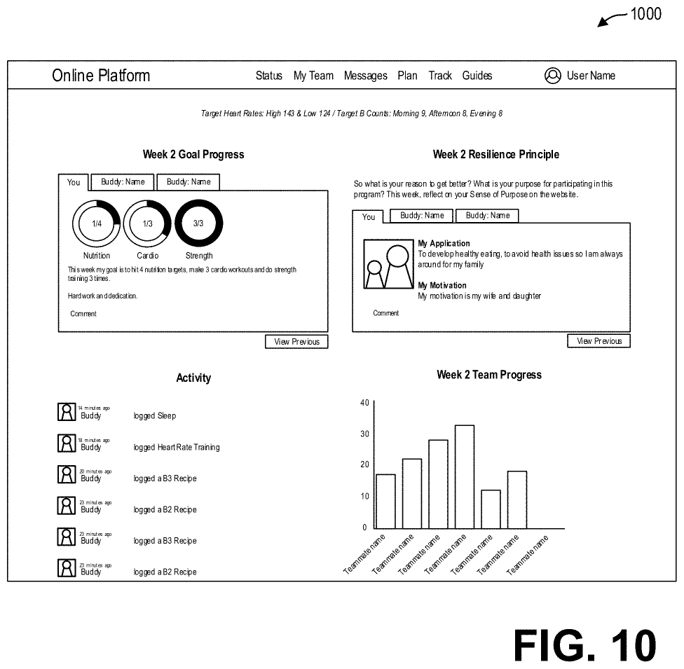

FIG. 10 shows an example interface 1000 for displaying a detailed overview of weekly progress towards personal targets for both a user and the user's team members and/or buddies. Interface 1000 may display information regarding goal progress, resilience principles, buddy activity, and overall team progress.

Interface 1000 may display each target goal category as a circle, for example, with a percentage of the circle's circumference highlighted corresponding to the number of targets achieved in that target category. As shown, 2/11ths of the nutrition target category is highlighted in order to convey that the user has logged 2 nutrition achievements while the user has set 11 nutrition targets as a goal. The entire cardio target category circle is highlighted in order to convey that the user has logged 2 cardio workouts while the user has set 2 cardio workouts as a goal. Similarly, half of the strength target category circle is highlighted in order to convey that the user has logged 1 strength achievement while the user has set 2 strength targets as a goal for the week. Note that interface 1000 may further include a tab for one or more team partners, or buddies, so that a user may view similar detailed information for the weekly progress of a team partner. In this way, a user may experience the mutually competitive and supportive attitude familiar to the user from military training.

Interface 1000 may display resilience principle information, such as a motivational image, a user-input motivation, and a user-input motivation. Interface 1000 may include such resilience principle information in order to provide a reminder of the user's motivation for adhering to the behavioral fitness program. Interface 1000 may further include a tab for one or more buddies so that a user may view similar resilience principle information for a team partner. In this way, a user may be develop a healthy mindfulness practice and stay motivated towards his or her goals.

Interface 1000 may further include an activity log for the user and the user's buddies. In this way, a user can view a detailed activity timeline and see that the user and/or other users are adhering to the program. Interface 1000 displays all activity logged by the user, including detailed workout information, meals consumed, and the time each activity occurred. Interface 1000 may further include a commenting feature so that the user and other stakeholders may comment on each logged activity. In this way, online platform 143 may function as social media, meaning that users may interact with logged activity of their own as well as other users. For example, a coach or a team member may user interface 1000 to comment on a meal or a workout logged by a user. In this way, the user experience of working towards his or her fitness goals is similar to the experience of military life.

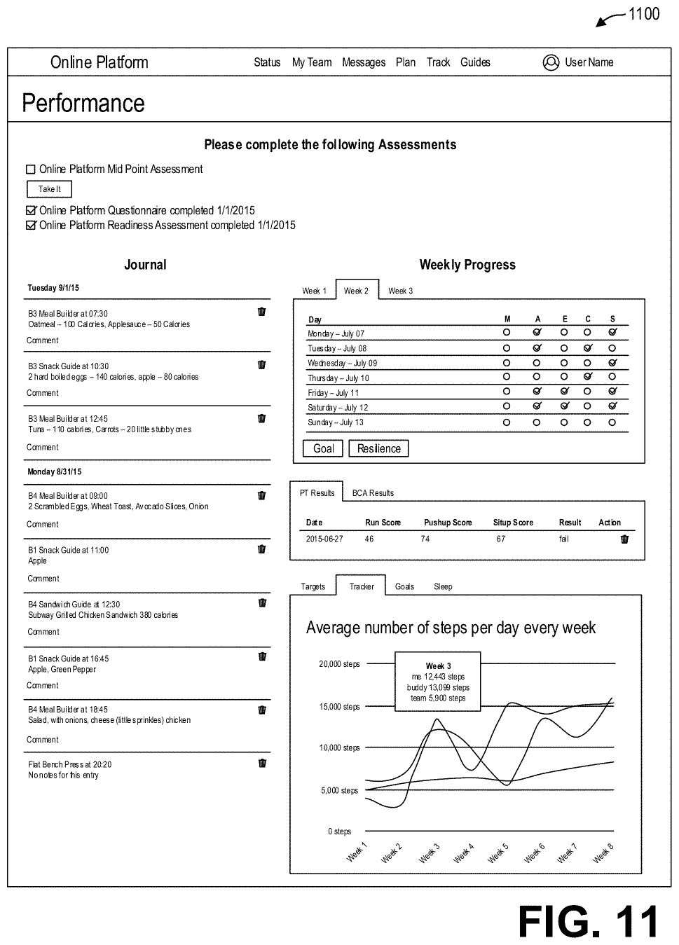

FIG. 11 shows an example interface 1100 for a detailed overview of weekly progress for a user. Interface 1100 may display information regarding complete or incomplete self-assessments, detailed weekly progress towards goals, a detailed overview of a user's journal, and weekly physical activity. For example, a user may use a pedometer to track the number of steps every day and upload such data to the server 110. Interface 1100 may then display, for example, the average number of steps per day.

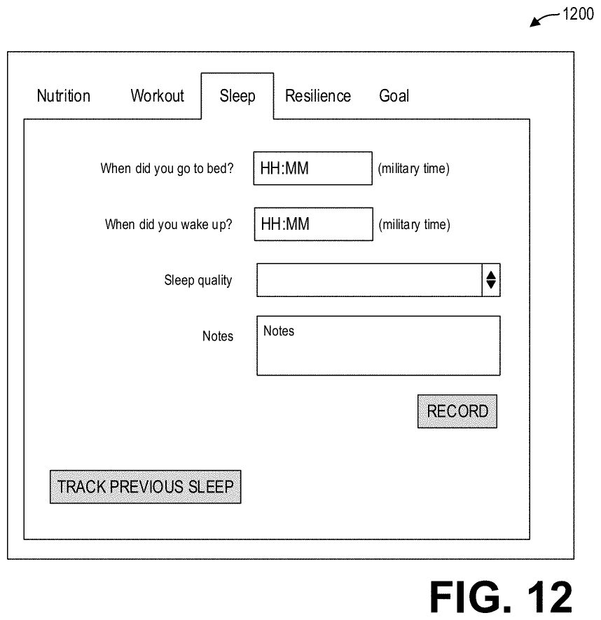

FIG. 12 shows an example interface 1200 for logging sleep information. Interface 1200 may include user inputs for data regarding sleep, such as when the user went to bed, when the user woke up, the quality of the sleep, and any notes regarding sleep. Server 110 may use data input to calculate total hours slept. In this way, a user can track their actual progress towards sleep goals. For example, if a user sets a goal of getting eight hours of good sleep every night, interface 1200 provides a simple way to determine if such goals are being achieved. In one example, sleep quality may be scored on a scale, for example from 1-4.

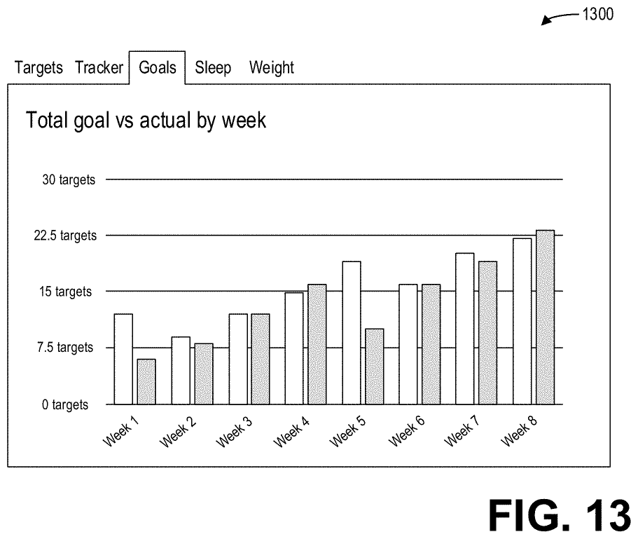

FIG. 13 shows an example interface 1300 that visually depicts target goals versus actual performance. In particular, interface 1300 displays a bar graph comparing goals targeted and actual goals achieved per week. In this way, online platform 143 may present an at-a-glance view of progress success.

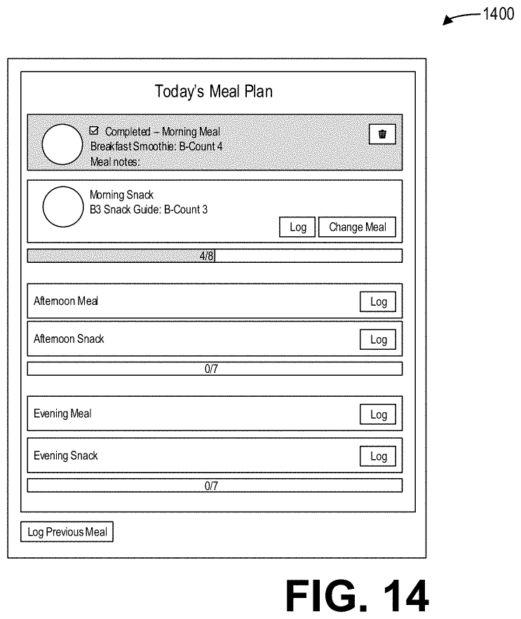

FIG. 14 shows an example interface 1400 for a daily meal plan in accordance with the current disclosure. In particular, interface 1400 shows an interactive daily meal plan log. Interface 1400 shows the three time sections—morning, afternoon, and evening—and target B-Counts for each section. In the example shown, the target B-Count for morning is 8, the target B-Count for afternoon is 7, and the target B-count for evening is 7. Interface 1400 displays what meals and snacks have been logged, including the B-Count and any meal notes input by the user. In the example shown, the user has logged the completed consumption of a morning meal comprising a breakfast smoothie with a B-Count of 4, and an intended consumption of a morning snack with a B-Count of 3. Note that interface 1400 further includes an option to log previous meals and an option for editing logged meals.

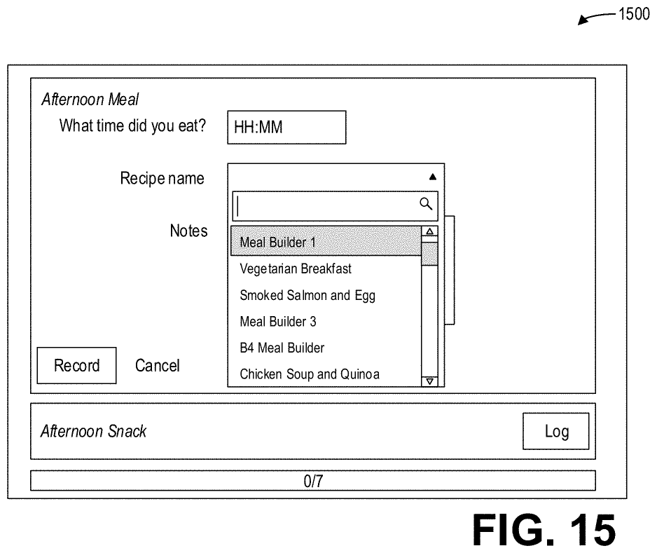

In order to log a meal, a user may click on a “Log” button corresponding to the meal or snack the user would like to log. FIG. 15 shows an example interface 1500 for logging a meal. In particular, interface 1500 depicts input fields displayed after clicking a “Log” button on interface 1400. As shown, interface 1500 may include a time field for inputting the time a meal or snack was consumed, a drop-down list containing all balanced meal recipes stored on server 110, and a text field for meal notes.

In some examples, server 110 includes an interactive repository of balanced meal recipes that may be accessed via online platform 143. Users may search recipes by name, ingredients, cuisine, and B-Count. Users may view meal recipes, add reviews of a meal recipe, and add a recipe for a balanced meal.

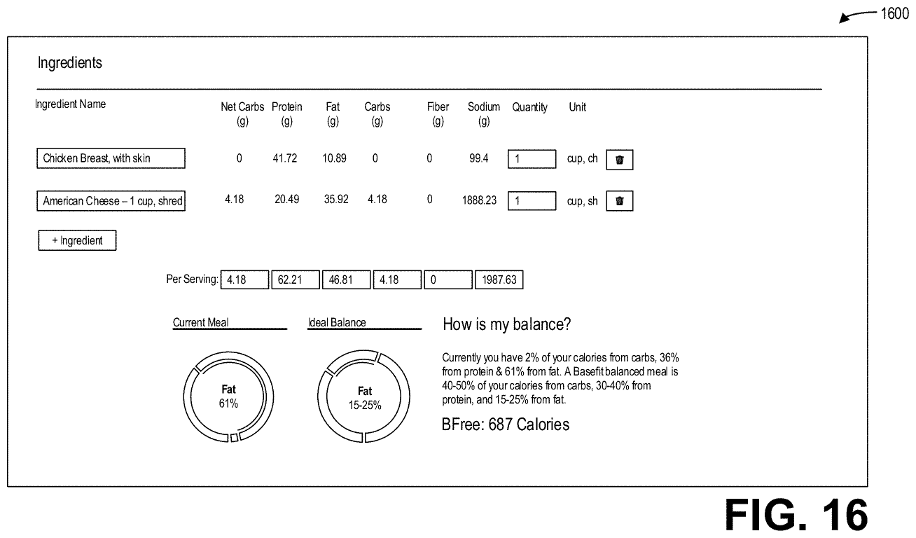

FIG. 16 shows an example interface 1600 for creating a recipe for a balanced meal in accordance with the current disclosure. A user may select ingredients for a recipe, and server 110 displays the ingredient on interface 1600 along with nutritional information from nutrition database 121. As shown, nutritional information may include, but is not limited to, net carbohydrates, protein, fat, carbohydrates, fiber, and sodium. The quantity of each ingredient may be adjusted according to the recipe. Server 110 computes overall nutritional value of the meal and shows a breakdown of the nutritional content. Interface 1600 may further include an interactive visual depiction of the nutritional balance for the recipe in addition to a visual depiction of an ideal nutritional balance. In the example shown, a recipe comprising one cup of chicken breast with skin and one cup of shredded American cheese contains 61% fat, 36% protein, and 2% carbohydrates. The meal balance and the ideal balance are depicted in a circular form with each component—fat, protein, and carbohydrates—forming a percentage of the circumference. Interface 1600 may further include an overall B-Count computed by server 110 using nutrition information from nutrition database 221.

After building a meal recipe using interface 1600, a user may save the recipe on server 110 so that the recipe may be accessed via online platform 143. In this way, the user may easily access the recipe when logging meals, share the recipe with other users, and receive comments on the specific meal recipe.

In some examples, a user may submit a recipe to the interactive recipe repository on server 110 without using interface 1600. For example, a user may write a recipe in his or her paper-based journal and submit a photograph of the journal entry containing the recipe. Server 110 may then process the image, interpret the recipe, and generate a B-Count for the recipe using nutritional information from nutrition database 221. In another example, a user may send a message, such as a text message or a picture message, containing the recipe. Server 110 may then process the message, interpret the recipe, and generate a B-Count for the recipe using nutritional information from nutrition database 221. In another example, a user may find a recipe while searching on the Internet and may like to know the B-Count for the recipe. Thus, server 110 may include an interface for users to submit web pages containing recipes. Server 110 may interpret the recipe on the web page and generate a B-Count for the recipe. Such an interface may take the form of a web page on online platform 143, or may, for example, take the form of a browser plug-in that allows the user to click a button to automatically process a recipe on a web page. In all examples, server 110 may include an interface similar to interface 1600 that displays the B-Count of a recipe to a user. In this way, a user may determine the B-Count of a recipe with minimal effort.

In some examples, the server 110 may include one or more interfaces enabling a user to select weekly motivational images and resilience principles. Such an interface may display encouraging information regarding resilience principles. A user may reflect upon this information and choose a motivational image that embodies the resilience principles displayed. To that end, the interface may further enable a user to upload a weekly motivational image that may displayed to the user and other team members each week. Further, the interface may include inputs for a user to describe how the user will implement the weekly resilience principle, the motivation for a user to implement the weekly resilience principle, and a weekly motivational image.

FIG. 17 shows an example interface 1700 for displaying weekly motivational images. Interface 1700 may display an array of one or more weekly motivational images that reflect the resilience principles displayed via the interface described herein above, for example. Interface 1700 may be accessible to each member of a team, and may include motivational images submitted by each team member. In some examples, interface 1700 may display motivational images submitted by all users in a user's location. Each motivational image may comprise a photograph of, for example, each user's family, a motivational message, and so on. In this way, users may view the motivational images of similar users, thereby developing cohesion between the users.

Interface 1700 may further include a brief overview of weekly physical goals set by the user. In this way, a user may contemplate the goals set while viewing the motivational images that he or she submitted.

In some examples, the server 110 may include one or more interfaces that enable direct communication between team members and a coach. Secure messages may be exchanged via server 110 between team members and a coach. In this way, a user may directly communicate in private with a coach and/or one or more team members via online platform 143.

In other examples, the server 110 may include one or more interfaces that enable open communication between team members and a coach. Such an interface may comprise a team page for a particular team. On a team page, users may post text, images, links to external sites, videos, and comments. In some examples, a team page may be private such that only users who are members of the team may visit the team page. In this way, team members may openly communicate and exchange information with one another.

The server 110 may further include one or more interfaces that display aggregated information for a leader, such as a coach or other stakeholders. FIG. 18 shows an example interface 1800 for a leadership dashboard. The leadership dashboard offers a real-time measurement of all user interactions through the online platform, activity monitor data streams, and personal health coaching measurements. The dashboard delivers actionable reporting to engage with users in their training earlier and more effectively when compared to traditional aggregate reporting, thereby improving training delivery, personalization, and adherence. The primary metric of the interface 1800 is a qualified measurement of participant engagement based on multiple criteria and health coach interactions. The user engagement score rates individual participants on their personal engagement in the online platform training experience using a three-point scale: engaged, active, and disengaged. Engaged participants embrace the training, show strong participation during team sessions, and actively execute lifestyle changes. Active participants grasp specific aspects of the training, attend some of the team sessions, and are overall ready to make a change. Disengaged participants display limited understanding of the training, do not participate in team sessions, have little interaction with coaches, and do not show a willingness to change. The engagement metric used is a combination of quantitative data points gathered from the online platform, combined with a qualitative measure by health coaching staff based on in-person and telephonic interactions.

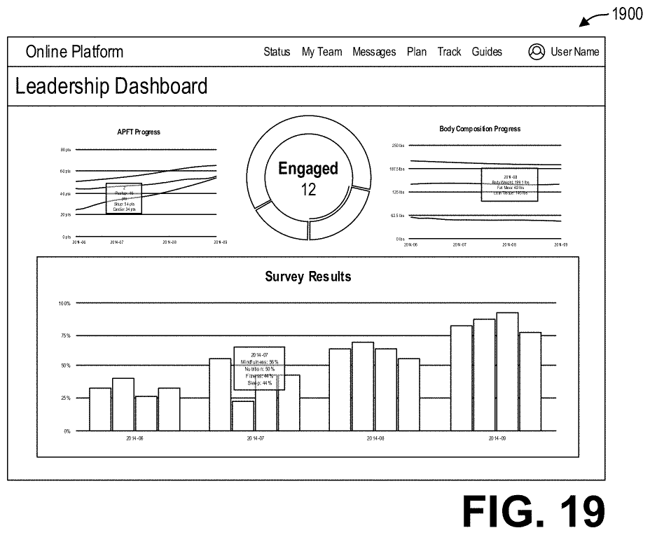

Dashboard contents displayed in interface 1800 are available in real-time and access is dictated by specific access levels assigned to each stakeholder. Further, aggregate survey and assessment response data are made available for long-term behavioral trend analysis. FIG. 19 shows an example interface 1900 for displaying aggregated qualitative metrics to show long-term health behavior modification and trends.

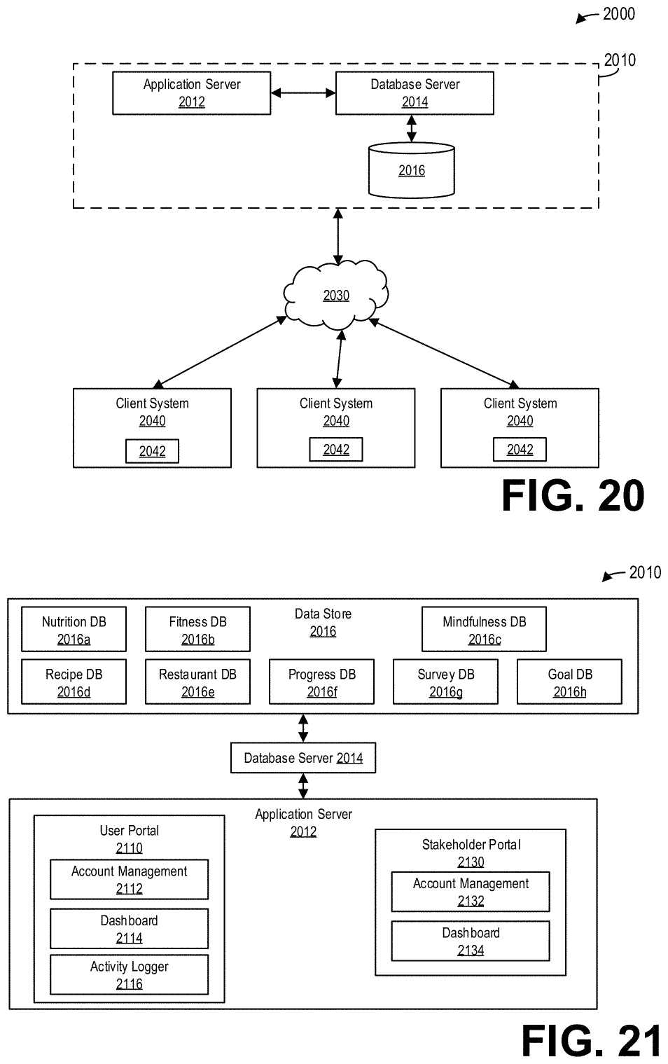

Referring now to FIG. 20, a schematic diagram illustrating an example network architecture for a behavioral fitness achievement system 2000 that can be configured to implement exemplary embodiments of the present invention, such as those described herein above, is provided. It should of course be understood that the behavioral fitness achievement system 2000 is intended as an example, not as an architectural limitation for different embodiments of the present invention, and therefore, the particular elements depicted in FIG. 20 should not be considered limiting with regard to the environments within which exemplary embodiments of the present invention may be implemented.

In the example illustrated in FIG. 20, behavioral fitness achievement system 2000 is implemented as a client/server system that includes a central server system 2010 that is commonly accessed by each user of the system through operation of any of a plurality of client systems 2040 that are operatively coupled to the central server system via a communication network 2030. Central server system 2010 further includes a database server 2014 that is coupled to a data store 2016 and an application server 2012, and each client system 2040 is a user terminal or other client device implementing software for running a respective client application 2042 for accessing services provided via a network-based application (also referred to herein as a network service) implemented by application server 2012. Such client applications may also be referred to as client modules, or simply clients, and may be implemented in a variety of ways. In exemplary embodiments, such client applications can be implemented as any of a myriad of suitable client application types, which range from proprietary client applications to web-based interfaces in which the application functionality is provided by a web server and/or a back-end program.

In exemplary embodiments, the behavioral fitness achievement system 2000 can include additional servers, clients, and other devices not shown in FIG. 20. The particular architecture depicted in FIG. 20 is provided as an example for illustrative purposes and, in exemplary embodiments, any number of client systems 2040 may be connected to server system 2010 at any given time via network 2030, and server system 2010 can comprise multiple server components and databases located within a single server system or within multiple server systems, where the multiple server systems are integrated with or accessible by users of client systems 2040 as a distributed server system via network 2030.

In exemplary embodiments, network 2030 can be configured to facilitate communications between server system 2010 and client systems 2040, as well as communications with and between other devices and computers connected together within the behavioral fitness achievement system 2000, by any suitable wired technology (including optical fiber), wireless technology, or any suitable combination thereof, including but not limited to personal area networks (PANs), local area networks (LANs), wireless networks, wide-area networks (WANs), the Internet (a network of heterogeneous networks using the Internet Protocol), and virtual private networks, and the network may also utilize any suitable hardware, software, and firmware technology to connect devices such as, for example, optical fiber, Ethernet, ISDN (Integrated Services Digital Network), T-1 or T-3 link, FDDI (Fiber Distributed Data Network), cable or wireless LIVIDS network, Wireless LAN, Wireless PAN (for example, IrDA, Bluetooth, Wireless USB, Z-Wave, Zig-Bee), HomePNA, power line communication, or telephone line network. Such a network connection can include intranets, extranets, and the Internet, may contain any number of network infrastructure elements including routers, switches, gateways, etc., can comprise a circuit switched network, such as the Public Service Telephone Network (PSTN), a packet switched network, such as the global Internet, a private WAN or LAN, a telecommunications network, a broadcast network, or a point-to-point network, and may utilize a variety of networking protocols now available or later developed including, but not limited to the Transmission Control Protocol/Internet Protocol (TCP/IP) suite of protocols for communication.

In exemplary embodiments, application server 2012, database server 2014, and any other servers employed within server system 2010 and third-party servers utilized within behavioral fitness achievement system 2000 can be implemented within any suitable computing system or systems such as a workstation computer, a mainframe computer, a server system, a server cluster, a distributed computing system, a cloud-based computing system, or the like, as well as any of the various types of computing systems and devices described herein. Server system 2010 may be implemented using any of a variety of architectures. For example, application server 2012 and database server 2014 may also be implemented independently or as a single, integrated device. While the exemplary embodiment illustrated in FIG. 20 depicts application server 2012 and database server 2014 as individual components, the applications provided by these servers, or various combinations of these applications, may actually be server applications running on separate physical devices. In this regard, server system 2010 may comprise a number of computers connected together via a network, and therefore, may exist as multiple separate logical and/or physical units, and/or as multiple servers acting in concert or independently, wherein each server may be comprised of multiple separate logical and/or physical units. In exemplary embodiments, server system 2010 can be connected to network 2030 through a collection of suitable security appliances, which may be implemented in hardware, software, or a combination thereof.

As illustrated in FIG. 20, application server 2012 is communicatively coupled to database server 2014. Database server 2014 is connected to data store 2016, which comprises a plurality of databases that are maintained by database server 2014, accessed by application server 2012 via database services provided at a front end by database server 2014, and store information on a variety of matters that is utilized in providing the services offered via the network service provided by the application server, as described herein. As used herein, the term “data store,” “data storage unit,” “storage device,” and the like can refer to any suitable memory device that may be used for storing data, including manual files, machine-readable files, and databases. In exemplary embodiments, application server 2012, database server 2014, and data store 2016 may be implemented together in a single computing device, implemented within a plurality of computing devices locally coupled to each other via a suitable communication medium, such as a serial port cable, telephone line or wireless frequency transceiver, implemented within a plurality of computing devices remotely coupled to each other via network 2030, or any suitable combination thereof.

Client systems 2040 are computer devices to which one or more users, which may be users of the network service as described herein above (including typical users and stakeholders). It should be noted that the term “user” is used herein to refer to one who uses a computer system, such as one of the client systems 2040. As described herein, client systems 2040 are each operable by such users to access server system 2010 via network 2030 and act as clients to access services offered by the network service provided by the server system within exemplary behavioral fitness achievement system 2000. For this purpose, each client system includes a respective client application 2042 that executes on the client system and allows a user to interact with server system 2010 via application server 2012.

In exemplary embodiments, the computer systems of client systems 2040 can be any of a wide range of suitable computing devices such as one or more workstations, desktop computers, laptops, or other personal computers (PCs), non-traditional computer systems such as personal digital assistants (PDAs) and other handheld or portable electronic devices, smart phones and other mobile handsets, tablet computers, netbook computers, game consoles, home theater PCs, desktop replacement computers, and the like, or any other suitable information processing devices.

In general, during operation of exemplary behavioral fitness achievement system 2000, a client system 2040 first establishes a connection to server system 2010 via network 2030. Once the connection is established, the connected client system may directly or indirectly transmit data to and access content from the application server 2012. A user accessing application server 2012 through the connected client system can use client application 2042 to access services provided by the application server, which are described in detail herein above, via a user interface implemented by the client application within which the client application renders the information served by the application server.

In exemplary embodiments, application server 2012 can implement the network service as a non-web client application (such as a mobile application), a web client application, or both to provide the services accessed by client systems 2040 within server system 2010, and client applications 2042 can correspondingly be implemented as non-web client applications, web client applications, or both for operation by users of the client systems to interact with application server 2012 and access the services provided thereby. For example, application server 2012 can comprise a web server configured to provide a web application for the respective client applications implemented on client systems 2040 that are configured to provide web-based user interfaces for utilizing the services provided by the web server. For instance, the user interfaces of client applications implemented on client systems 2040 can be configured to provide various options corresponding to the functionality offered in exemplary embodiments described herein through suitable user interface controls (for example, by way of menu selection, point-and-click, dialog box, or keyboard command). In one general example, the user interfaces may provide “send” or “submit” buttons that allow users of client applications to transmit requested information to application server 2012. The user interfaces can be implemented, for example, as a graphical user interface (GUI) that renders a common display structure to represent the network service provided by application server 2012 for a user of a client platform.

More specifically, in such an example, application server 2012 can, for example, be configured to provide services via a web-based software application hosting a corresponding website that includes a number of web pages (e.g., screens), and client applications 2042 can comprise a web browser executing on client systems 2040, such that the services provided by application server 2012 are accessible to client systems 2040 using the Internet or an intranet. Users of client systems 2040 may thereby access the website provided by application server 2012 by, for example, inputting or following a link to the uniform resource locator (URL) for the website in the web browser, which then enable users to display and interact with information, media, and other content embedded within the web pages of the website provided by application server 2012. The web-based software application can transmit information that can be processed by the web browsers to render a user interface using, for example, a browser-supported programming language such as JavaScript, HTML, HTML5, CSS, or the like, and can communicate with the web browsers using, for example, HTTPS, POST, and/or GET requests. Client applications 2042 and application server 2012 may be configured such that information transmitted between client systems 2040 and server system 2010 can be encrypted and sent over a secure network connection to protect privacy.

Referring now to FIG. 21, a block diagram illustrating an exemplary embodiment of server system 2010 is provided. As illustrated in FIG. 21, application server 2012 is implemented to provide a plurality of services via a user portal 2110 and a plurality of services via a stakeholder portal 2130. As described herein, application server 2012 can be implemented to provide a respective set of services for each of various types of users (for example, users, stakeholders), and some of the services offered by application server 2012 can be commonly applicable to and accessible by all types of users, while other services can be applicable to and accessible only by specific types of users. The services provided via user portal 2110 includes a registration and account management service 2112, a dashboard service 2114, and an activity logger 2116, while the services provided via stakeholder portal 2130 includes a registration and account management service 2132 and dashboard service 2134.

Data store 2016 comprises a plurality of databases that are maintained and accessible by application server 2012 via database server 2014, including a nutrition database 2016a (as described herein above with regard to FIG. 2), a fitness database 2016b (as described herein above with regard to FIG. 2), a mindfulness database 2016c (as described herein above with regard to FIG. 2), a recipe database 2016d (as described herein above with regard to FIGS. 15-16), a restaurant database 2016e (as described herein above with regard to FIGS. 8-7), a progress database 2016f (for storing user activity data regarding progress towards goals), a survey database 2016g (for storing user responses to qualitative surveys and assessments), and a health balance goal database 2016h (for storing health balance goals established by users and coaches). The data store 2016 may include additional databases not shown for storing any other suitable information that may be utilized by server system 2010 (for example, system usage data). In exemplary embodiments, the various databases maintained within data store 2016 can be maintained as groups within one or more larger databases or maintained individually.

Thus, in one embodiment, an apparatus for achieving and maintaining behavioral fitness comprises: a server providing a network service that is accessible to a plurality of users through a plurality of client systems communicatively coupled to the server via a network; and a data storage system storing a health balance goal database that is maintained by the server, the health balance goal database comprising a plurality of health balance goals established by the plurality of users, and wherein, upon being accessed by a user of one of the client systems, the network service is operable to receive an indication from the client system of a selected health balance goal selected from the plurality of health balance goals by the user, wherein, upon receiving the selection of the selected health balance goal from the client system, the network service is operable to issue a request for approval of the selected health balance goal by a coach via one of the client systems, and wherein the network service is operable to, upon receiving the approval from the coach: generate a meal plan, a workout plan, and a mindfulness plan based on the selected health balance goal, store the meal plan, the workout plan, and the mindfulness plan in the health balance goal database, receive data indicating one or more user activities related to one or more of the meal plan, the workout plan, and the mindfulness plan, store the received data within a progress database that is maintained within the data storage system by the server, and transmit a user interface to the client system for display to the user, the user interface generated based on the received data and visually depicting user progress towards meeting the health balance goal, the user progress based on the one or more user activities.

In a first example of the apparatus, the health balance goal comprises a combination of a fitness goal, mindfulness goal, and nutrition goal. In a second example of the apparatus optionally including the first example, the meal plan comprises a target B-Count for each meal, wherein a B-Count comprises a combination of caloric content and macronutrient content. In a third example of the apparatus optionally including one or more of the first and second examples, the network service is further operable to, responsive to receiving a restaurant query from the client device: detect a location of the user; calculate a current target B-Count based on the meal plan and a time the restaurant query is received; retrieve at least one result from a restaurant database that is maintained within the data storage system by the server based on the location of the user and the current target B-Count, wherein the at least one result comprises a restaurant and at least one menu item corresponding to the current target B-Count; and transmit the at least one result to the client device. In a fourth example of the apparatus optionally including one or more of the first through third examples, the at least one result is displayed to the user via a map, wherein the map further indicates the location of the user. In a fifth example of the apparatus optionally including one or more of the first through third examples, the network service is further operable to: receive a list of one or more ingredients for a meal; calculate a B-Count and a macronutrient balance of the meal based on the list of the one or more ingredients; and generate a user interface including a visual depiction of the macronutrient balance of the meal positioned adjacent to a visual depiction of an ideal macronutrient balance, and the calculated B-Count. In a sixth example of the apparatus optionally including one or more of the first through fifth examples, receiving the data related to the one or more activities comprises receiving, from the user device, an image of a paper-based journal entry containing the data in a handwritten format, and automatically processing the image to extract the data from the image. In a seventh example of the apparatus optionally including one or more of the first through sixth examples, the user interface includes a form comprising a plurality of user inputs regarding sleep, the plurality of user inputs including a bed time, a wake-up time, and a sleep quality, and further comprising updating the user progress responsive to the user submitting the form and based on the plurality of user inputs. In an eighth example of the apparatus optionally including one or more of the first through seventh examples, the network service is further operable to adjust one or more of the meal plan, the fitness plan, and the mindfulness plan responsive to the user progress. In a ninth example of the apparatus optionally including one or more of the first through eighth examples, the user interface includes at least one graph visually comparing the user progress to progress of one or more other users.

In another representation, a method comprises: receiving, from a user via a user device, a health balance goal for the user; receiving, from a coach via a coach device, approval of the health balance goal; automatically generating a meal plan, a workout plan, and a mindfulness plan based on the health balance goal; receiving, from the user via the user device, data indicating one or more user activities related to one or more of the meal plan, the workout plan, and the mindfulness plan; and automatically generating a user interface for display via one or more of the user device and the coach device, the user interface visually depicting user progress towards meeting the health balance goal, the user progress based on the one or more user activities.

In a first example of the method, the health balance goal comprises a combination of a fitness goal, mindfulness goal, and nutrition goal. In a second example of the method optionally including the first example, the meal plan comprises a target B-Count for each meal, wherein a B-Count comprises a combination of caloric content and macronutrient content. In a third example of the method optionally including one or more of the first and the second examples, the method further comprises, responsive to receiving a restaurant query from the user device: detecting a location of the user; calculating a current target B-Count based on the meal plan and a time the restaurant query is received; retrieving at least one result based on the location of the user and the current target B-Count, wherein the at least one result comprises a restaurant and at least one menu item corresponding to the current target B-Count; and transmitting the at least one result to the user device. In a fourth example of the method optionally including one or more of the first through third examples, the method further comprises displaying the at least one result on a map, wherein the map further indicates the location of the user. In a fifth example of the method optionally including one or more of the first through fourth examples, the method further comprises: receiving a list of one or more ingredients for a meal; calculating a B-Count and a macronutrient balance of the meal based on the list of the one or more ingredients; and automatically generating a user interface including a visual depiction of the macronutrient balance of the meal positioned adjacent to a visual depiction of an ideal macronutrient balance, and the calculated B-Count. In a sixth example of the method optionally including one or more of the first through fifth examples, receiving the data regarding the one or more activities comprises receiving, from the user device, an image of a paper-based journal entry containing the data in a handwritten format, and automatically processing the image to extract the data from the image. In a seventh example of the method optionally including one or more of the first through sixth examples, the user interface includes a form comprising a plurality of user inputs regarding sleep, the plurality of user inputs including a bed time, a wake-up time, and a sleep quality, and further comprising updating the user progress responsive to the user submitting the form and based on the plurality of user inputs. In an eighth example of the method optionally including one or more of the first through seventh examples, the method further comprises adjusting one or more of the meal plan, the fitness plan, and the mindfulness plan responsive to the user progress. In a ninth example of the method optionally including one or more of the first through eighth examples, the user interface includes at least one graph visually comparing the user progress to progress of one or more other users.

In another embodiment, a computer-generated interface for display to a user on a user device comprises: a visual overview of individual progress of the user with regard to nutrition goals and fitness goals; a graph visually depicting the individual progress of the user in comparison to other users; one or more resilience principles and a personal motivation input by the user; and an activity log displaying recently logged progress for the user and the other users.

In a first example of the interface, the interface further comprises a plurality of user inputs for logging progress towards the nutrition goals and the fitness goals. In a second example of the interface optionally including the first example, the plurality of user inputs for logging progress towards the nutrition goals comprises one or more user inputs for selecting a recipe from a list of recipes retrieved from a repository, wherein the repository further includes data regarding caloric content and macronutrient content for each meal. In a third example of the interface optionally including one or more of the first and second examples, the nutrition goals and the fitness goals are established based on the user and a coach of the user. In a fourth example of the interface optionally including one or more of the first through third examples, the personal motivation comprises an image uploaded by the user.

In yet another embodiment, a computer-readable storage medium includes executable instructions stored thereon, the executable instructions configured to cause a computer to: automatically assign a category to each user of a plurality of users based on quantitative and qualitative data; aggregate the quantitative and qualitative data received from the plurality of users; generate a user interface based on the aggregated quantitative and qualitative data, including a graph illustrating a number of users in the plurality of users assigned to each category; and transmit the user interface to a computing device for display to a stakeholder.

In a first example of the computer-readable storage medium, the aggregated quantitative and qualitative data comprises survey results received from the plurality of users, and wherein the user interface includes a graph comparing the survey results over time. In a second example of the computer-readable storage medium optionally including the first example, the user interface further includes a graph illustrating aggregated fitness test results over time. In a third example of the computer-readable storage medium optionally including one or more of the first and the second examples, the user interface further includes a graph illustrating body composition progress over time, wherein the body composition progress comprises data regarding body weight, fat mass, and lean tissue mass. In a fourth example of the computer-readable storage medium optionally including one or more of the first through third examples, the user interface further includes an overview of goals and progress towards the goals for individual users in the plurality of users based on the category assigned to the individual users.

In yet a further embodiment that may optionally include one or more of the previously discussed examples a method, comprises receiving a restaurant query from a device for a user meal plan; detecting GPS coordinates of the device; locating one or more restaurants that are less than a threshold distance from the device based on the detected GPS coordinates; calculating a current target B-Count that is personalized for the user meal plan; determining at least one restaurant menu item of the one or more restaurants is within the current target B-Count for the user; and displaying the at least one restaurant menu item and displaying a B-Count value associated with each of the at least one restaurant menu items, wherein the B-Count value is a value that indicates a predetermined macronutrient balance and a caloric content. It is noted that systems may further include instructions stored in non-transitory memory of one or more devices (e.g., a user device, a server, etc.) to carry out the methods. In a first example of the embodiment, the B-Count value may be an aggregated value calculated based on macronutrients and caloric content. In a second example, which optionally includes the first example, determining the at least one restaurant menu item of the one or more restaurants is within the current target B-Count for the user may include comparing nutritional information for the current target B-Count and the at least one restaurant menu item while the current target B-Count and the at least one restaurant menu item are in B-Count format, wherein the B-Count format is a format where the nutritional information is represented by B-Count values. In a third example which optionally includes one or both of the first and second examples, the predetermined macronutrient balance may be a predetermined ratio of macronutrients relative to one another that is personalized to the meal plan of the user. In a fourth example, which optionally includes one or more of the first through third examples, the current target B-Count for the user is based on a current time of day. In a fifth example, which optionally includes one or more of the first through fourth examples, the current target B-Count for the user may be further based on meals tracked as consumed. In a sixth example, which optionally includes one or more of the first through fifth examples, the at least one restaurant menu item of the one or more restaurants may be within the current target B-Count for the user includes converting menus of the one or more restaurants from restaurant menu format to B-Count format. In a seventh example, which optionally includes one or more of the first through sixth examples, converting the menus of the one or more restaurants to B-Count format may include analyzing the restaurant menus to determine a macronutrient content and a caloric content for each of the one or more restaurant menu items, and calculating the B-Count for each of the one or more restaurant menu items, wherein the B-Count may be an aggregated value calculated from the macronutrient content and the caloric content for each of the one or more restaurant menu items.

In a further embodiment, which optionally includes one or more of the previously discussed examples, a system, comprises a network service device comprising instructions stored in non-transitory memory that are executable to: receive a restaurant query from a device for a user; locate one or more restaurants within a threshold distance of the device; calculate a current nutritional target for the user, the current nutritional target represented by an aggregated value, the aggregated value calculated using macronutrient meal plan targets and caloric meal plan targets for the user; determine at least one restaurant menu item of the one or more restaurants is within the current nutritional target for the user; and display the at least one restaurant menu item and an aggregated value for each of the at least one restaurant menu items, wherein the aggregated value displayed for each of the at least one restaurant menu items is calculated using macronutrient values and caloric values for each of the at least one restaurant menu items. In a first example of the embodiment, the one or more restaurants may be located as being within the threshold distance of the device based on GPS coordinates detected for the device, the GPS coordinates detected upon receipt of the restaurant query. In a second example of the embodiment, which optionally includes the first example, displaying the at least one restaurant menu item may include displaying a location of a restaurant serving the at least one restaurant menu item and a location of the device on a map. In a third example of the embodiment, which optionally includes one or both of the first and second examples, displaying the at least one restaurant menu item may include displaying one or more modifications for the at least one restaurant menu item. In a fourth example of the embodiment, which may optionally include one or more of the first through third examples, the one or more modifications include at least one of additions, a request to withhold one or more ingredients, and cooking instructions. In a fifth example of the embodiment, which may optionally include one or more of the first through fourth embodiments, the aggregated value for each of the at least one restaurant menu items may be a B-Count value.

In yet an even further embodiment, which may optionally include one or more of any of the examples described herein, a system comprises a network service device comprising instructions stored in non-transitory memory that are executable to: receive a restaurant query from a user device for a user meal plan; detect GPS coordinates of the user device; based on a current time of day when the restaurant query is received and the GPS coordinates of the user device, determine at least one restaurant menu item that is within a current target B-Count for the user meal plan; and display the at least one restaurant menu item and a B-Count value associated with the at least one restaurant menu item as part of a geolocation restaurant guide, wherein the B-Count value is a value that indicates a predetermined macronutrient balance and a caloric content. In a first example of the embodiment, the current target B-Count may be time-based and varies depending on both the current time of day and meals recorded by a user for a day. In a second example of the embodiment, which optionally includes the first example, the current target B-Count is further varied depending on exercise recorded by the user for the day. In a third example of the embodiment, which optionally includes one or both of the first and second examples, the instructions are further executable to display a location of one or more restaurants associated with the at least one restaurant menu and a location of the user device on a map. In a fourth example of the embodiment, which optionally includes one or more of the first through third examples, the at least one restaurant menu item and the B-Count value associated with the at least one restaurant menu item may be further displayed on the map. In a fifth example of the embodiment, which optionally includes one or more of the first through fourth examples, the at least one restaurant menu item is selectable to indicate that the at least one restaurant menu item is being consumed.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
receiving a restaurant query from a device for a user meal plan;
detecting GPS coordinates of the device;
locating one or more restaurants that are less than a threshold distance from the device based on the detected GPS coordinates;
calculating a current target B-Count that is personalized for the user meal plan;
determining at least one restaurant menu item of the one or more restaurants is within the current target B-Count for the user; and
displaying the at least one restaurant menu item and displaying a B-Count value associated with each of the at least one restaurant menu items as part of a gelocation restaurant menu guide, wherein the B-Count value is a value that indicates a predetermined macronutrient balance and a caloric content.

2. The method of claim 1, wherein the B-Count value is an aggregated value calculated based on macronutrients and caloric content.

3. The method of claim 2, wherein determining the at least one restaurant menu item of the one or more restaurants is within the current target B-Count for the user includes comparing nutritional information for the current target B-Count and the at least one restaurant menu item while the current target B-Count and the at least one restaurant menu item are in B-Count format, wherein the B-Count format is a format where the nutritional information is represented by B-Count values.

4. The method of claim 3, wherein the predetermined macronutrient balance is a predetermined ratio of macronutrients relative to one another that is personalized to the meal plan of the user.

5. The method of claim 1, wherein the current target B-Count for the user is based on a current time of day.

6. The method of claim 5, wherein the current target B-Count for the user is further based on meals tracked as consumed.

7. The method of claim 1, wherein determining the at least one restaurant menu item of the one or more restaurants is within the current target B-Count for the user includes converting menus of the one or more restaurants from restaurant menu format to B-Count format.

8. The method of claim 7, wherein converting the menus of the one or more restaurants to B-Count format includes analyzing the restaurant menus to determine a macronutrient content and a caloric content for each of the one or more restaurant menu items, and calculating the B-Count for each of the one or more restaurant menu items, wherein the B-Count is an aggregated value calculated from the macronutrient content and the caloric content for each of the one or more restaurant menu items.

9. A system, comprising:
a network service device comprising instructions stored in non-transitory memory that are executable to:
receive a restaurant query from a device for a user;
locate one or more restaurants within a threshold distance of the device;
calculate a current nutritional target for the user, the current nutritional target represented by an aggregated value, the aggregated value calculated using macronutrient meal plan targets and caloric meal plan targets for the user;
determine at least one restaurant menu item of the one or more restaurants is within the current nutritional target for the user; and
display the at least one restaurant menu item and an aggregated value for each of the at least one restaurant menu items, wherein the aggregated value displayed for each of the at least one restaurant menu items is calculated using macronutrient values and caloric values for each of the at least one restaurant menu items.

10. The system of claim 9, wherein the one or more restaurants are located as being within the threshold distance of the device based on GPS coordinates detected for the device, the GPS coordinates detected upon receipt of the restaurant query.

11. The system of claim 10, wherein displaying the at least one restaurant menu item includes displaying a location of a restaurant serving the at least one restaurant menu item and a location of the device on a map.

12. The system of claim 9, wherein displaying the at least one restaurant menu item includes displaying one or more modifications for the at least one restaurant menu item.

13. The system of claim 12, wherein the one or more modifications include at least one of additions, a request to withhold one or more ingredients, and cooking instructions.

14. The system of claim 9, wherein the aggregated value for each of the at least one restaurant menu items is a B-Count value.

15. A system, comprising:
a network service device comprising instructions stored in non-transitory memory that are executable to:
receive a restaurant query from a user device for a user meal plan;
detect GPS coordinates of the user device;
based on a current time of day when the restaurant query is received and the GPS coordinates of the user device, determine at least one restaurant menu item that is within a current target B-Count for the user meal plan; and
display the at least one restaurant menu item and a B-Count value associated with the at least one restaurant menu item as part of a geolocation restaurant guide, wherein the B-Count value is a value that indicates a predetermined macronutrient balance and a caloric content.

16. The system of claim 15, wherein the current target B-Count is time-based and varies depending on both the current time of day and meals recorded by a user for a day.

17. The system of claim 16, wherein the current target B-Count is further varied depending on exercise recorded by the user for the day.

18. The system of claim 15, wherein the instructions are further executable to display a location of one or more restaurants associated with the at least one restaurant menu and a location of the user device on a map.

19. The system of claim 18, wherein the at least one restaurant menu item and the B-Count value associated with the at least one restaurant menu item are further displayed on the map.

20. The system of claim 19, wherein the at least one restaurant menu item is selectable to indicate that the at least one restaurant menu item is being consumed.
